(19) United States
(12) Patent Application Publication
(10) Pub. No.: US 2003/0235916 A1
(43) Pub. Date: Dec. 25, 2003

(54) Novel methods for the delivery of polynucleotides to cells

(76) Inventors: Sean D. Monahan, Madison, WI (US); Lisa Nader, Madison, WI (US); Jon A. Wolff, Madison, WI (US); Vladimir G. Budker, Middleton, WI (US); James E. Hagstrom, Middleton, WI (US)

Correspondence Address: Mark K. Johnson, Mirus Corporation, 505 S. Rosa Rd., Madison, WI, 53719, US

(21) Appl. No.: 10/462,138

(22) Filed: Jun. 16, 2003

Related U.S. Application Data
(60) Provisional application No. 60/388,685, filed on Jun. 14, 2002.

(51) Int. Cl. A61K 48/00; C12N 15/85

(52) U.S. Cl. 435/455; 514/44

(57) ABSTRACT

Process are described for the delivery of a polynucleotide to a cell. The process comprises forming a salt stable complex between the polynucleotide and a cationic surfactant. Ternary complexes are also made by associating an amphipathic compound with the binary complex. The resultant complexes are suitable for delivery of the polynucleotide to cells in vitro and in vivo.

### CROSS-REFERENCE TO RELATED APPLICATIONS

This application is related to prior provisional application U.S. Serial No. 60/388,685 filed Jun. 14, 2002.

### FIELD OF THE INVENTION

The technical field of the present invention is a composition comprising polynucleotide, amphipathic compounds and polymers, and the use of such compositions for delivering the polynucleotide to an animal cell.

### BACKGROUND

Gene And Nucleic Acid-Based Delivery—Gene or polynucleotide transfer to cells is an important technique for biological and medical research as well as potentially therapeutic applications. The polynucleotide needs to be transferred across the cell membrane and into the cell. For polynucleotides encoded expressible genes, the polynucleotide must be delivered to the cell nucleus where the gene can be transcribed. Gene transfer methods currently being explored include viral vectors and non-viral methods.

Viruses have evolved over millions of year to transfer their genes into mammalian cells. Viruses can be modified to carry a desired gene and become a “vector” for gene delivery. Using recombinant techniques, harmful or superfluous viral genes can be removed and replaced with a desired gene. This technique was first accomplished with mouse retroviruses. The development of retroviral vectors is being pursued for gene therapy applications. However, these vectors cannot infect all cell types efficiently, especially in vivo. Therefore, several viral vectors, including Herpes virus, Adenovirus, Adeno-associated virus and others are being developed to enable more efficient gene transfer different cell types, including brain and lung.

Non-viral vectors are also being developed to transfer polynucleotides into mammalian cells. For these non-viral vectors, an expressible gene is typically cloned into a plasmid that is capable of replicating in bacteria, usually E. coli. The desired gene is recombinantly inserted into the bacterial plasmid along with a mammalian promoter, enhancer, or other sequences that enable the gene to be expressed in mammalian cells. Milligrams of the plasmid DNA can then be prepared and purified from bacterial cultures. Alternatively, polynucleotides for delivery to cells can by made enzymatically such as by PCR, or they can be synthesized chemically. The polynucleotides can be incorporated into lipid vesicles (liposomes including cationic lipids such as Lipofectin) which transfer the polynucleotide into the target mammalian cell. Polynucleotides can also be complexed with polymers such as polylysine, polyethylenimine, and proteins. Other methods of polynucleotide delivery to cells include electroporation and “gene gun” technologies.

Gene delivery approaches can be classified into direct and indirect methods. Some of these gene transfer methods are most effective when directly injected into a tissue space. Direct methods using many of the above gene transfer techniques are being used to target tumors, muscle, liver, lung, and brain. Other methods are most effective when applied to cells or tissues that have been removed from the body and the genetically-modified cells are then transplanted back into the body. Indirect approaches in conjunction with retroviral vectors are being developed to transfer genes into bone marrow cells, lymphocytes, hepatocytes, myoblasts and skin cells.

Gene Therapy And Nucleic Acid-Based Therapies—Gene therapy promises to be a revolutionary advancement in the treatment of disease. With gene therapy, a disease state can be directly treated by inserting a corrective polynucleotide into cells. In contrast, traditional drug based approaches act downstream on the products of the genes (proteins, enzymes, enzyme substrates and enzyme products). Although, the initial motivation for gene therapy was the treatment of genetic disorders, it is becoming increasingly apparent that gene therapy will be useful for the treatment of a broad range of acquired diseases such as cancer, infectious diseases, heart disease, arthritis, and neurodegenerative disorders (such as Parkinson's and Alzheimer's).

In addition to providing an exogenous gene, gene therapy also has the potential to inhibit endogenous genes. Several mechanisms exist for specifically inhibiting expression of an endogenous gene. These include antisense nucleic acid, ribozymes, and small inhibitory RNA (siRNA) mediated RNA interference (RNAi). Antisense inhibition involves single stranded polynucleotide that is complementary to the target mRNA. Ribozymes are catalytic RNAs capable of specifically cleaving a target mRNA. SiRNAs are short double stranded RNAs that are identical in sequence to a segment of the expressed target gene and, in conjunction with cellular proteins, cause the degradation of the target RNA. Specific inhibition of endogenous gene expression has great potential in the treatment of dominant genetic disorders, viral infections, and cancer

Gene transfer can also be used as a vaccination against infectious diseases and cancer. When a foreign gene is transferred to a cell and expressed, the resultant protein is presented to the immune system. This presentation differs from the antigen presentation resulting from simply injecting the protein into the body and is more likely to cause a cell-mediated immune response. Expression of the viral gene within a cell simulates a viral infection without the danger of an actual viral infection and induces a more effective immune response. This approach may be more effective in for fighting latent viral infections such as human immunodeficiency virus, Herpes and cytomegalovirus.

Polymers for Nucleic Acid Delivery—Polymers have been used in research for the delivery of polynucleotides to cells. One of the several methods of polynucleotide delivery to cells is the use of polynucleotides/polycation complexes. It has been shown that cationic proteins, like histones and protamines, or synthetic polymers, like polylysine, polyarginine, polyornithine, DEAE dextran, polybrene, and polyethylenimine may be effective intracellular polynucleotide delivery agents. The following are some important principles involving the mechanism by which polycations facilitate uptake of polynucleotides:

Polycations facilitate nucleic acid condensation. The volume which one polynucleotide molecule occupies in a complex with polycations is drastically lower than the volume of the free polynucleotide molecule. The size of a polynucleotides/polymer complex is probably critical for gene delivery in vivo and possible for in vitro as well. For intravascular delivery, the polynucleotide needs to cross the endothelial barrier in order to reach the parenchymal cells of interest. The largest endothelial fenestrae (holes in the endothelial barrier) occur in the liver and have an average diameter of 100 nm. The trans-epithelial pores in other organs are much smaller. For example, muscle endothelium can be described as a structure which has a large number of small pores with a radius of 4 nm, and a very low number of large pores with a radius of 20-30 nm. The size of the polynucleotide complexes is also important for the cellular uptake process. After binding to the cells the polynucleotide/polycation complex is likely taken up by endocytosis. Since endocytic vesicles have a typical internal diameter of about 100 nm, polynucleotide complexes smaller than 100 nm are preferred.

Polycations may provide attachment of polynucleotides to the cell surface. The polymer forms a cross-bridge between the polyanionic polynucleotide and the polyanionic surface of a cell. As a result, the mechanism of polynucleotide translocation to the intracellular space might be non-specific adsorptive endocytosis. This process may be more effective than either fluid phase endocytosis or receptor-mediated endocytosis. Furthermore, polycations provide a convenient linker for attaching specific ligands to the complex. The polynucleotide/polycation complexes could then be targeted to specific cell types.

The polynucleotides in polycation complexes are protected against nuclease degradation. This protection is important for both extra- and intracellular preservation of polynucleotide since nucleases are present in serum and endosomes/lysosomes. Protection from degradation in endosomes/lysosomes is enhanced by preventing organelle acidification. One method of preventing acidification is the use of NH₄Cl or chloroquine. Other polymers, such as polyethylenimine, probably disrupt endosomal/lysosomal function without additional treatments. Disruption of endosomal/lysosomal function has also been accomplished by linking endosomal or membrane disruptive agents such as fusion peptides or adenoviruses to the polycation or complex.

Condensation of nucleic acid—A significant number of multivalent cations with widely different molecular structures have been shown to induce(condensation of nucleic acid. Multivalent cations with a charge of three or higher have been shown to condense DNA. These include spermidine, spennine, Co(NH₃)₆³⁺,Fe³⁺, and natural or synthetic polymers such as histone H1, protamine, polylysine, and polyethylenimine. Analysis has shown DNA condensation to be favored when 90% or more of the charges along the sugar-phosphate backbone are neutralized. Neutral and anionic polymers can increase repulsion between DNA and its surroundings, therefore compacting the DNA. Most significantly, spontaneous DNA self-assembly and aggregation processes have been shown to result from the confinement of large amounts of DNA due to excluded volume effect.

The mechanism of polynucleotide condensation is not clear. The electrostatic force between unperturbed helices arises primarily from a counter-ion fluctuation mechanism requiring multivalent cations and plays a major role in polynucleotide condensation. The hydration forces predominate over electrostatic forces when the nucleic acid helices approach closer then a few water diameters. In a case of DNA/polymeric polycation interactions, DNA condensation is a more complicated process than the case of low molecular weight polycations. Different polycationic proteins can generate toroid and rod formation with different size DNA at a ratio of positive to negative charge of two to five. DNA complexes with polyarginine or histone can form two types of structures; an elongated structure with a long axis length of about 350 nm (like free DNA) and dense spherical particles. Both forms exist simultaneously in the same solution. The reason for the co-existence of the two forms can be explained as an uneven distribution of the polycation chains among the DNA molecules. The uneven distribution generates two thermodynamically favorable conformations.

The electrophoretic mobility of polynucleotide/polycation complexes can change from negative to positive in excess polycation. It is likely that large polycations do not completely align along the polynucleotide but form polymer loops which interact with other polynucleotide molecules. The rapid aggregation and strong intermolecular forces between different nucleic acid molecules may prevent the slow adjustment between helices needed to form tightly packed orderly particles.

Surface charging—As discussed previously, polycations can help adhere polynucleotide complexes to a cell surface. However, negative surface charge would be more desirable for many practical applications, i.e. in vivo delivery. The phenomenon of surface recharging is well known in colloid chemistry and is described in great detail for lyophobic/lyophilic systems (i.e., silver halide hydrosols). Addition of polyion to a suspension of latex particles with an oppositely-charged surface leads to the permanent absorption of the polyion onto the surface. Upon reaching appropriate stoichiometry, the surface is changed to the opposite charge.

The Use of pH-Sensitive Lipids, Aniphipathic Conipounds, and Liposomes for Nucleic Acid Delivery—Cationic liposomes may deliver DNA either directly across the plasma membrane or via the endosome compartment. Regardless of its exact entry point, much of the DNA within cationic liposomes accumulates in the endosome compartment. Several approaches have been investigated to prevent loss of the foreign DNA in the endosomal compartment by protecting it from hydrolytic digestion or enabling its escape into the cytoplasm. These approaches include the use of acidotropic (lysomotrophic) weak amines, such as chloroquine, which presumably prevent DNA degradation by inhibiting endosomal acidification [Legendre et al. 1992]. Alternatively viruses and viral fusion peptides have been included to disrupt endosomes or promote fusion of liposomes with endosomes and facilitate release of DNA into the cytoplasm [Kamata et al. 1994; Wagner et al. 1994)].

Knowledge of lipid phases and membrane fusion has been used to design potentially more versatile liposomes which exploit endosomal acidification to promote fusion with endosomal membranes. Such an approach is best exemplified by anionic, pH-sensitive liposomes that have been designed to destabilize or fuse with the endosome membrane at acidic pH [Duzgunes et al. 1991)]. All of the anionic, pH-sensitive liposomes have utilized phosphatidylethanolamine (PE) bilayers that are stabilized at non-acidic pH by the addition of lipids that contain a carboxylic acid group. Liposomes containing only PE are prone to the inverted hexagonal phase (H_{II}). In pH-sensitive, anionic liposomes, the carboxylic acid's negative charge increases the size of the lipid head group at pH greater than the carboxylic acid's PKₐ and thereby stabilizes the phosphatidylethanolamine bilayer. At acidic pH conditions found within endosomes, the uncharged or reduced charge species is unable to stabilize the phosphatidylethanolamine-rich bilayer. Anionic, pH-sensitive liposomes have delivered a variety of membrane-impermeable compounds including DNA. However, the negative charge of these pH-sensitive liposomes prevents them from efficiently taking up DNA and interacting with cells; thus decreasing their utility for transfection. We have described the use of cationic, pH-sensitive liposomes to mediate the efficient transfer of DNA into a variety of cells in culture U.S. Ser. No. 08/530,598, and U.S. Ser. No. 09/020,566.

The Use of pH-Sensitive Polymers for Nucleic Acid Delivery—Polymers that are pH-sensitive have found broad application in the area of drug delivery because of their ability to exploit various physiological and intracellular pH gradients for the purpose of controlled release of drugs. pH sensitivity can be broadly defined as any change in polymer's physico-chemical properties over a range of pH. Narrower definitions demand significant changes in the polymer's ability to retain or release a bioactive substance in a physiologically tolerated pH range (typically pH 5.5-8). All polyions can be divided into three categories based on their ability to donate or accept protons in aqueous solutions: polyacids, polybases and polyampholytes. Use of pH-sensitive polyacids in drug delivery applications usually relies on their ability to a) become soluble with a pH increase (acid/salt conversion), b) form a complex with other polymers over a change of pH, or c) undergo significant change in hydrophobicity/hydrophilicity balance. Combinations of all three above factors are also possible.

### SUMMARY OF THE INVENTION

In a preferred embodiment we describe an in vivo process for delivering a polynucleotide to a cell comprising: associating the polynucleotide with a cationic surfactant in an aqueous solution to form a complex, stabilizing the complex, and bringing the complex into contact with the cell. Stabilizing the complex comprises: incubating the complex in the aqueous solution at elevated temperature. The incubation may be from several minutes to hours, depending on the temperature. Alternatively, stabilizing the complex comprises: drying the complex, dissolving the complex in an appropriate organic solvent, and diluting with an appropriate aqueous solution. A stable particle comprises condensed polynucleotide wherein the size of the complex does not rapidly increase nor does the polynucleotide rapidly decondense if the complex is exposed to salt at physiological concentrations. Bringing the complex into contact with the cell may comprise: directly injecting the complex in an aqueous solution into a tissue or inserting the complex in an aqueous solution into a vessel-in a mammal for delivery to cell in a tissue to which the vessel either supplies or drains a bodily fluid.

In a preferred embodiment, we describe a process for forming a stable polynucleotide/cationic surfactant complex comprising: associating the polynucleotide with the cationic surfactant in an aqueous solution and incubating the solution at elevated temperature. The incubation may be from several minutes to hours, depending on the temperature. The elevated temperature may be 30° C. to 100° C. or higher. More preferably, the elevated temperature is 35° C. to 50° C. A stable particle comprises condensed polynucleotide wherein the size of the complex does not rapidly increase nor does the polynucleotide rapidly decondense if the complex is exposed to physiological salt concentrations.

In a preferred embodiment, we describe a process for delivering a polynucleotide to a cell comprising: associating a polynucleotide with a cationic surfactant to form a binary complex, associating the complex with an amphipathic compound to form a ternary complex, and associating the complex with a cell. The amphipathic compound may be selected from the list comprising: polymers containing hydrophobic moieties, peptides containing hydrophobic moieties, targeting groups containing hydrophobic moieties, steric stabilizers containing hydrophobic moieties, surfactants and lipids. The amphipathic compound may be cationic, anionic, neutral, or zwitterionic. The resultant ternary complex can have a net surface charge that is positive, negative or neutral. The amphipathic compound may also be modified to contain one or more functional groups that increase transfection efficiency. The amphipathic compound may be modified prior to ternary complex formation of after ternary complex formation. The binary complex may be stabilized prior to formation of the ternary complex.

Stabilizing the binary complex comprises: incubating the complex in aqueous solution at elevated temperature. The incubation may be from several minutes to hours, depending on the temperature. Alternatively, stabilizing the complex comprises: drying the complex, dissolving the complex in an appropriate organic solvent, and diluting with an appropriate aqueous solution. A stable particle comprises condensed polynucleotide wherein the size of the complex does not rapidly increase nor does the polynucleotide rapidly decondense if the complex is exposed to salt at physiological concentrations. The ternary complex may be delivered to a cell in vivo or in vitro. Delivering the ternary complex to a cell in vivo may comprise: directly injecting the complex in an aqueous solution into a tissue or inserting the complex in an aqueous solution into a vessel in a mammal for delivery to cell in a tissue to which the vessel either supplies or drains a bodily fluid.

In a preferred embodiment, we describe a process for forming small, less than 50 nm diameter, polynucleotide-containing complexes comprising: associating a polynucleotide with a cationic surfactant to form a binary complex, stabilizing the binary complex, and associating the binary complex with an amphipathic compound to form a ternary complex. Stabilizing the binary complex comprises: incubating the complex in aqueous solution at elevated temperature. The incubation may be from several minutes to hours, depending on the temperature. Alternatively, stabilizing the complex comprises: drying the complex, dissolving the complex in an appropriate organic solvent, and diluting with an appropriate aqueous solution. A stable particle comprises condensed polynucleotide wherein the size of the complex does not rapidly increase nor does the polynucleotide rapidly decondense if the complex is exposed to salt at physiological concentrations. The amphipathic compound may be selected from the list comprising: polymers containing hydrophobic moieties, peptides containing hydrophobic moieties, targeting groups containing hydrophobic moieties, steric stabilizers containing hydrophobic moieties, surfactants and lipids, The amphipathic compound may be cationic, anionic, neutral, or zwitterionic. The resultant ternary complex can have a net surface charge that is positive, negative or neutral. The amphipathic compound may also be modified to contain one or more functional groups that increase transfection efficiency. The amphipathic compound may be modified prior to ternary complex formation of after ternary complex formation.

Further objects, features, and advantages of the invention will be apparent from the following detailed description when taken in conjunction with the accompanying drawings.

### BRIEF DESCRIPTION OF THE FIGURES

FIG. 1. Fluorescence of DNA/cationic surfactant complexes after ultracentrifugation.

FIG. 2. DNA/Cationic Surfactant Complex Degradation with DNAse I.

FIG. 3. RhDNA Condensation with Dodecylamine Hydrochloride.

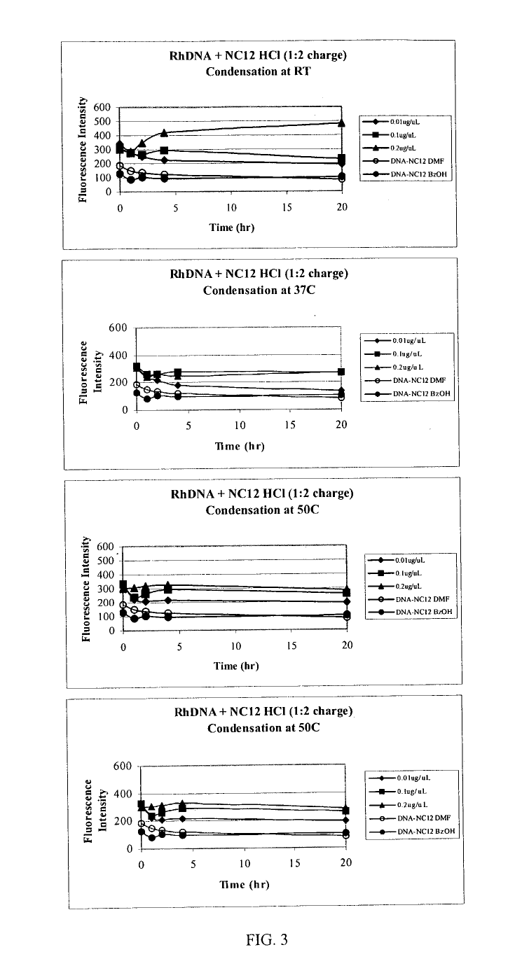

FIG. 4. RhDNA/Cationic Surfactant Complex Stability with NaCl.

FIG. 5. RhDNA-NC12 Stability at 37° C. or in 150 mM NaCl.

FIG. 6. RhDNA-NC12 Stability with 10% Serum.

FIG. 7. Circular Dichroism of DNA/Cationic Surfactant Complexes.

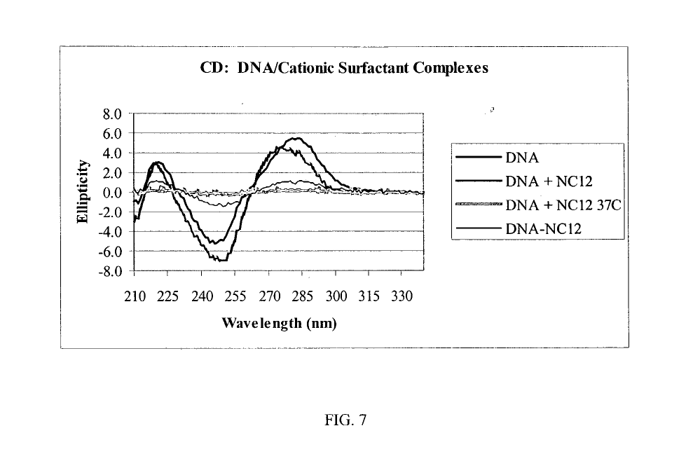

### DETAILED DESCRIPTION OF THE INVENTION

Several reports have been presented in the literature describing the formation of a complex between DNA and cationic surfactants [Melnikov et al. 1995a; Sergeyev et al. 1999a; Sergeyev et al. 1999b; Sukhorukov et al. 2000; Tanaka et al. 1996; Ijiro et al. 1992; Melnikoz et al. 1995b]. For example, Melnikov et al. showed a phase transition between random coil and compact globule states for DNA upon the addition of cetyltrimethylammonium bromide (CTAB). This complex formation was further shown to be reversible upon the addition of a competing polyanion (polyacrylic acid). The complexes can be prepared in such a way as to precipitate the complex. Upon thorough drying, the complexes can be solubilized in organic solvents such as ethanol, chloroform, THF, and cyclohexane. Although proposed in several of the reports, gene transfer has not been demonstrated with the DNA/cationic surfactant complexes. In fact, Clamme et al [Clamme et al. 2000] demonstrated that DNA/CTAB complexes were trapped onto the external face of the plasma membrane, thereby constituting a major limitation in efficient transfection.

Depending on the concentrations used, a precipitate may or may not form. The literature describes isolating the solid precipitate and dissolving the sample in organic solutions. We have found that the precipitated complex (DNA/cationic surfactant) and non-precipitated complex form inherently different complexes. Both complexes exhibit condensed DNA [Trubetskoy et al. 1999] and similar particle sizes. However, the complexes show vastly different stability, based on particle sizing and fluorescence decondensation assays, in the presence of physiological salt concentrations. The complex that is dried and taken up in organic solutions, shows stability to 150 mM NaCl without an increase in particle size. However, the non-precipitated complex is not stable in 150 mM NaCl, showing increasing particle size and decondensation. The present invention provides for the process by which stable complexes can be formed (indicated by salt stability assays, DNA condensation, and particle size stability) from non-precipitative concentrations of DNA and cationic surfactants. We show that DNA/cationic surfactant complexes can be formed in aqueous solutions, and heated at various temperatures for several minutes to hours, resulting in the formation of a complex that is salt stable. We further show that DNA/cationic surfactant complexes can be formed in aqueous solutions, lyophilized to dryness, dissolved in an appropriate organic solvent, and diluted with appropriate aqueous solutions resulting in the formation of a complex that is salt stable.

The present invention provides for the transfer of polynucleotides to cells in vivo and in vitro. Processes are described for the preparation and delivery of DNA/cationic surfactant complexes in vivo. Additionally, the present invention provides for the preparation and delivery of DNA/cationic surfactant/third component complexes in vivo and in vitro.

Although the literature indicates that DNA/cationic surfactant complexes do not transfect cells in vitro, we have found that the complexes are capable of DNA delivery in vivo. Intrvascular injection can be accomplished by dissolving the complex in an organic solvent and diluting the sample with an appropriate aqueous buffer prior to injection. Studies indicate that the complex remains a particle in the solution and that the particle is able to transfect cells. Mouse tail vein injection can also be accomplished by forming the DNA/cationic surfactant complex in an aqueous buffer, heating the sample for an amount of time, and diluting the sample with an appropriate aqueous buffer prior to injection. Studies indicate that the complex remains a particle in the solution and that the particle is able to transfect cells.

Additionally, the DNA/cationic surfactant complex can be mixed with polymers containing hydrophobic moieties to form a new complex. The new ternary complexes increase transfections in vivo relative to the DNA/cationic surfactant complex, and are also functional for the transfection of the DNA to cells in vitro. Complex formation with the hydrophobically modified polymers can occur below the critical micelle concentration (cmc) of the polymer. The polymer can be either a polycation or a polyanion and can contain targeting groups. Additional components can be added to the ternary complex, such as but not limited to, additional polymers (including charge modified polymers), peptides, hydrophobically modified peptides, charge modified peptides, amphipathic compounds, anionic surfactants, membrane active compounds, and salts.

Additionally, the DNA/cationic surfactant complex can be mixed with peptides containing hydrophobic moieties to form a new complex. The new ternary complexes can be used for cell transfections in vivo and in vitro. Complex formation with the hydrophobically modified peptides can occur below the critical micelle concentration (cmc) of the hydrophobically modified peptide. The peptide can be net cationic, net anionic, or net neutral charge. Additional components can be added to the ternary complex, such as but not limited to, polymers (including charge modified polymers), peptides, hydrophobically modified peptides, charge modified peptides, amphipathic compounds, anionic surfactants, membrane active compounds, and salts.

Additionally, the DNA/cationic surfactant complex can be mixed with targeting groups containing hydrophobic moieties to form a new complex. The new ternary complexes can be used for cell transfections in vivo and in vitro. Complex formation with the hydrophobically modified targeting group can occur below the critical micelle concentration (cmc) of the hydrophobically modified targeting group. The hydrophobically modified targeting group can be net cationic, net anionic, or net neutral charge. Additional components can be added to the ternary complex, such as but not limited to, polymers (including charge modified polymers), peptides, hydrophobically modified peptides, charge modified peptides, amphipathic compounds, anionic surfactants, membrane active compounds, and salts.

Additionally, the DNA/cationic surfactant complex can be mixed with steric stabilizers containing hydrophobic moieties to form a new complex. The new ternary complexes can be used for cell transfections in vivo and in vitro. Complex formation with the hydrophobically modified steric stabilizer can occur below the critical micelle concentration (cmc) of the hydrophobically modified steric stabilizer. The hydrophobically modified targeting group can be net cationic, net anionic, or net neutral charge. Additional components can be added to the ternary complex, such as but not limited to, polymers (including charge modified polymers), peptides, hydrophobically modified peptides, charge modified peptides, amphipathic compounds, anionic surfactants, membrane active compounds, and salts.

Additionally, the DNA/cationic surfactant complex can be mixed with anionic surfactants to form a new complex. The new ternary complexes can be used for cell transfections in vivo and in vitro. Complex formation with the anionic surfactants can occur below the critical micelle concentration (cmc) of the anionic surfactants. Additional components can be added to the ternary complex, such as but not limited to, polymers (including charge modified polymers), peptides, hydrophobically modified peptides, charge modified peptides, amphipathic compounds, membrane active compounds, and salts.

In another preferred embodiment, a process is described in that the DNA/cationic surfactant complexes can combined with amphipathic compounds, evaporated into a film, hydrated to from complex containing liposomes. The complexes can contain additional components including but not limited to polymers (including charge modified polymers, hydrophobically modified polymers), peptides, hydrophobically modified peptides, charge modified peptides, amphipathic compounds, anionic surfactants, membrane active compounds, and salts.

Definitions:

Complex—Two molecules are combined to form a complex through a process called complexation or complex formation if the are in contact with one another through noncovalent interactions such as electrostatic interactions, hydrogen bonding interactions, or hydrophobic interactions.

Binary complex—A binary complex is meant to include the complex formed between a polynucleotide and a cationic surfactant. The cationic surfactant can be a single surfactant, a mixture of surfactants (positive, negative, and neutral) with a net overall charge of positive, or one or more surfactants with one or more other components, such as a salt.

Ternary complex—A ternary complex is the complex formed when one or more components is added to a binary complex.

Elevated Temperature—The term elevated temperature in ment to mean temperatures greater than 25° C. and less than 100° C. Elevated temperature also means the combination of temperature and time that affords salt stability to a binary complex.

Polynucleotide—The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1methyladenine, 1methylpseudo-uracil, 1methylguanine, 1methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

DNA may be in form of cDNA, in vitro polymerized DNA, plasmid DNA, parts of a plasmid DNA, genetic material derived from a virus, linear DNA, vectors (PI, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 21-25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a gene(s). The term recombinant as used herein refers to a polynucleotide molecule that is comprised of segments of polynucleotide joined together by means of molecular biological techniques. The cassette contains the coding region of the gene of interest along with any other sequences that affect expression of the gene. A DNA expression cassette typically includes a promoter (allowing transcription initiation), and a sequence encoding one or more proteins. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences.

The polynucleotide may contain sequences that do not serve a specific function in the target cell but are used in the generation of the polynucleotide. Such sequences include, but are not limited to, sequences required for replication or selection of the polynucleotide in a host organism.

A polynucleotide can be used to modify the genomic or extrachromosomal DNA sequences. This can be achieved by delivering a polynucleotide that is expressed. Alternatively, the polynucleotide can effect a change in the DNA or RNA sequence of the target cell. This can be achieved by hybridization, multistrand polynucleotide formation, homologous recombination, gene conversion, or other yet to be described mechanisms.

The term gene generally refers to a polynucleotide sequence that comprises coding sequences necessary for the production of a therapeutic polynucleotide (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences influence the level or rate of transcription and translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, and immunogenicity. One example of covalent modification of nucleic acid involves the action of LabelIT reagents (Mirus Corporation, Madison, Wis.).

As used herein, the term gene expression refers to the process of converting genetic information encoded in a gene into RNA (e.g., mRNA, rRNA, tRNA, or snRNA) through transcription of a deoxyribonucleic gene (e.g., via the enzymatic action of an RNA polymerase), and for protein encoding genes, into protein through translation of mRNA. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

An RNA function inhibitor comprises any polynucleotide or nucleic acid analog containing a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. RNA function inhibitors are selected from the group comprising: siRNA, interfering RNA or RNAi, dsRNA, RNA Polymerase III transcribed DNAs encoding siRNA or antisense genes, ribozymes, and antisense nucleic acid, which may be RNA, DNA, or artificial nucleic acid. SiRNA comprises a double stranded structure typically containing 15-50 base pairs and preferably 21-25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The RNA function inhibitor may be polymerized in vitro, recombinant RNA, contain chimeric sequences, or derivatives of these groups. The RNA function inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

Transfection—The process of delivering a polynucleotide to a cell has been commonly termed transfection or the process of transfecting and also it has been termed transformation. The term transfecting as used herein refers to the introduction of a polynucleotide or other biologically active compound into cells. The polynucleotide may be used for research purposes or to produce a change in a cell that can be therapeutic. The delivery of a polynucleotide for therapeutic purposes is commonly called gene therapy. The delivery of a polynucleotide can lead to modification of the genetic material present in the target cell. The term stable transfection or stably transfected generally refers to the introduction and integration of an exogenous polynucleotide into the genome of the transfected cell. The term stable transfectant refers to a cell which has stably integrated the polynucleotide into the genomic DNA. Stable transfection can also be obtained by using episomal vectors that are replicated during the eukaryotic cell division (e.g., plasmid DNA vectors containing a papilloma virus origin of replication, artificial chromosomes). The term transient transfection or transiently transfected refers to the introduction of a polynucleotide into a cell where the polynucleotide does not integrate into the genome of the transfected cell. If the polynucleotide contains an expressible gene, then the expression cassette is subject to the regulatory controls that govern the expression of endogenous genes in the chromosomes. The term transient transfectant refers to a cell which has taken up a polynucleotide but has not integrated the polynucleotide into its genomic DNA.

Intravascular and vessel—The term intravascular refers to an intravascular route of administration that enables a polymer, oligonucleotide, or polynucleotide to be delivered to cells more evenly distributed than direct injections. Intravascular herein means within an internal tubular structure called a vessel that is connected to a tissue or organ within the body of an animal, including mammals. Vessels comprise internal hollow tubular structures connected to a tissue or organ within the body. Bodily fluid flows to or from the body part within the cavity of the tubular structure. Examples of bodily fluid include blood, lymphatic fluid, or bile. Examples of vessels include arteries, arterioles, capillaries, venules, sinusoids, veins, lymphatics, and bile ducts. Afferent blood vessels of organs are defined as vessels which are directed towards the organ or tissue and in which blood flows towards the organ or tissue under normal physiological conditions. Conversely, efferent blood vessels of organs are defined as vessels which are directed away from the organ or tissue and in which blood flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. Insertion of the inhibitor or inhibitor complex into a vessel enables the inhibitor to be delivered to parenchymal cells more efficiently and in a more even distribution compared with direct parenchymal injections.

Modification—A molecule is modified, to form a modification through a process called modification, by a second molecule if the two become bonded through a covalent bond. That is, the two molecules form a covalent bond between an atom form one molecule and an atom from the second molecule resulting in the formation of a new single molecule. A chemical covalent bond is an interaction, or bond, between two atoms in which there is a sharing of electron density. Modification also means an interaction between two molecules through a noncovalent bond. For example crown ethers can form noncovalent bonds with certain amine groups.

Salt—A salt is any compound containing ionic bonds; i.e., bonds in which one or more electrons are transferred completely from one atom to another. Salts are ionic compounds that dissociate into cations and anions when dissolved in solution and thus increase the ionic strength of a solution.

Pharmaceutically Acceptable Salt—Pharmaceutically acceptable salt means both acid and base addition salts.

Pharmaceutically Acceptable Acid Addition Salt—A pharmaceutically acceptable acid addition salt is a salt that retains the biological effectiveness and properties of the free base, is not biologically or otherwise undesirable, and is formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid, and the like.

Pharmaceutically Acceptable Base Addition Salt—A pharmaceutically acceptable base addition salt is a salts that retains the biological effectiveness and properties of the free acid, is not biologically or otherwise undesirable, and is prepared from the addition of an inorganic organic base to the free acid. Salts derived from inorganic bases include, but are not limited to, sodium, potassium, calcium, lithium, ammonium, magnesium, zinc, and aluminum salts and the like. Salts derived from organic bases include, but are not limited to, salts of primary secondary, and tertiary amines, such as methylamine, triethylamine, and the like.

Drying—Drying means removing the solvent from a sample, for example, removing the solvent from a complex under reduced pressure. Drying also means dehydrating a sample, or lyopholization of a sample.

Salt Stabilized Comtiplex—A salt stabilized complex is a complex that shows stability when exposed to 150 mM NaCl solution. Stability in this case is indicated by a stable particle size reading (less than a 20% change over 60 min )for the complex in 150 mM NaCl solution. Stability in this case is also indicated by no decondensation of the DNA (less than a 20% change over 60 min ) within the complex for the complex in 150 mM NaCl solution.

Interpolyelectrolyte Complexes—An interpolyelectrolyte complex is a noncovalent interaction between polyelectrolytes of opposite charge.

Charge, Polarity, and Sign—The charge, polarity, or sign of a compound refers to whether or not a compound has lost one or more electrons (positive charge, polarity, or sign) or gained one or more electrons (negative charge, polarity, or sign).

Functional group—Functional groups include cell targeting signals, nuclear localization signals, compounds that enhance release of contents from endosomes or other intracellular vesicles (releasing signals), and other compounds that alter the behavior or interactions of the compound or complex to which they are attached.

Cell targeting signals—Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

After interaction of a compound or complex with the cell, other targeting groups can be used to increase the delivery of the biologically active compound to certain parts of the cell.

Nuclear localization signals—Nuclear localizing signals enhance the targeting of the pharmaceutical into proximity of the nucleus and/or its entry into the nucleus during interphase of the cell cycle. Such nuclear transport signals can be a protein or a peptide such as the SV40 large T antigen NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus. For example, karyopherin beta itself could target the DNA to the nuclear pore complex. Several peptides have been derived from the SV40 T antigen. Other NLS peptides have been derived from the hnRNP A1 protein, nucleoplasmin, c-myc, etc. These include a short NLS (H-CGYGPKKKRKVGG-OH, SEQ ID 1) or long NLS's (H-CKKKSSSDDEATADSQHST-PPKKKRKVEDPKDFPSELLS-OH, SEQ ID 2 and H-CKKKWDDEATADSQHSTPPKKK-RKVEDPKDFPSELLS-OH, SEQ ID 3). Other NLS peptides have been derived from M9 protein (CYNDFGNYNNQSSNFGPMKQGNFGGRSSGPY, SEQ ID 4), EIA (H-CKRGPKRPRP-OH, SEQ ID 5), nucleoplasmin (H-CKKAVKRPAATKKAGQAKKKKL-OH, SEQ ID 6),and c-myc (H-CKKKGPAAKRVKLD-OH, SEQ ID 7).

Membrane active compounds—Many biologically active compounds, in particular large and/or charged compounds, are incapable of crossing biological membranes. In order for these compounds to enter cells, the cells must either take them up by endocytosis, i.e., into endosomes, or there must be a disruption of the cellular membrane to allow the compound to cross. In the case of endosomal entry, the endosomal membrane must be disrupted to allow for movement out of the endosome and into the cytoplasm. Either entry pathway into the cell requires a disruption or alteration of the cellular membrane. Compounds that disrupt membranes or promote membrane fusion are called membrane active compounds. These membrane active compounds, or releasing signals, enhance release of endocytosed material from intracellular compartments such as endosomes (early and late), lysosomes, phagosomes, vesicle, endoplasmic reticulum, golgi apparatus, trans golgi network (TGN), and sarcoplasmic reticulum. Release includes movement out of an intracellular compartment into the cytoplasm or into an organelle such as the nucleus. Releasing signals include chemicals such as chloroquine, bafilomycin or Brefeldin A1 and the ER-retaining signal (KDEL sequence), viral components such as influenza virus hemagglutinin subunit HA-2 peptides and other types of amphipathic peptides. The control of when and where the membrane active compound is active is crucial to effective transport. If the membrane active agent is operative in a certain time and place it would facilitate the transport of the biologically active compound across the biological membrane. If the membrane active compound is too active or active at the wrong time, then no transport occurs or transport is associated with cell rupture and cell death. Nature has evolved various strategies to allow for membrane transport of biologically active compounds including membrane fusion and the use of membrane active compounds whose activity is modulated such that activity assists transport without toxicity. Many lipid-based transport formulations rely on membrane fusion and some membrane active peptides' activities are modulated by pH. In particular, viral coat proteins are often pH-sensitive, inactive at neutral or basic pH and active under the acidic conditions found in the endosome.

Cell penetrating compounds—Cell penetrating compounds, which include cationic import peptides (also called peptide translocation domains, membrane translocation peptides, arginine-rich motifs, cell-penetrating peptides, and peptoid molecular transporters) are typically rich in arginine and lysine residues and are capable of crossing biological membranes. In addition, they are capable of transporting molecules to which they are attached across membranes. Examples include TAT (GRKKRRQRRR, SEQ ID 9), VP22 peptide, and an ANTp peptide (RQIKIWFQNRRMKWKK, SEQ ID 10). Cell penetrating compounds are not strictly peptides. Short, non-peptide polymers that are rich in amines or guanidinium groups are also capable of carrying molecules crossing biological membranes. Like membrane active peptides, cationic import peptides are defined by their activity rather than by strict amino acid sequence requirements.

Interaction Modifiers—An interaction modifier changes the way that a molecule interacts with itself or other molecules relative to molecule containing no interaction modifier. The result of this modification is that self-interactions or interactions with other molecules are either increased or decreased. For example cell targeting signals are interaction modifiers which change the interaction between a molecule and a cell or cellular component. Polyethylene glycol is an interaction modifier that decreases interactions between molecules and themselves and with other molecules.

Linkages—An attachment that provides a covalent bond or spacer between two other groups (chemical moieties). The linkage may be electronically neutral, or may bear a positive or negative charge. The chemical moieties can be hydrophilic or hydrophobic. Preferred spacer groups include, but are not limited to C1-C12 alkyl, C1-C12 alkenyl, C1-C12 alkynyl, C6-C18 aralkyl, C6-C18 aralkenyl, C6-C18 aralkynyl, ester, ether, ketone, alcohol, polyol, amide, amine, polyglycol, polyether, polyamine, thiol, thio ether, thioester, phosphorous containing, and heterocyclic. The linkage may or may not contain one or more labile bonds.

Bifunctional—Bifunctional molecules, commonly referred to as crosslinkers, are used to connect two molecules together, i.e. form a linkage between two molecules. Bifunctional molecules can contain homo or heterobifunctionality.

Labile Bond—A labile bond is a covalent bond that is capable of being selectively broken. That is, the labile bond may be broken in the presence of other covalent bonds without the breakage of the other covalent bonds. For example, a disulfide bond is capable of being broken in the presence of thiols without cleavage of other bonds, such as carbon-carbon, carbon-oxygen, carbon-sulfur, carbon-nitrogen bonds, which may also be present in the molecule. Labile also means cleavable.

Labile Linkage—A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals. The spacer group may contain chemical moieties chosen from a group that includes alkanes, alkenes, esters, ethers, glycerol, amide, saccharides, polysaccharides, and heteroatoms such as oxygen, sulfur, or nitrogen. The spacer may be electronically neutral, may bear a positive or negative charge, or may bear both positive and negative charges with an overall charge of neutral, positive or negative.

pH-Labile Linkages and Bonds—pH-labile refers to the selective breakage of a covalent bond under acidic conditions (pH<7). That is, the pH-labile bond may be broken under acidic conditions in the presence of other covalent bonds that are not broken.

Amphiphilic and Aniphipathic Compounds—Amphipathic, or amphiphilic, compounds have both hydrophilic (water-soluble) and hydrophobic (water-insoluble) parts.

Polymers—A polymer is a molecule built up by repetitive bonding together of smaller units called monomers. In this application the term polymer includes both oligomers which have two to about 80 monomers and polymers having more than 80 monomers. The polymer can be linear, branched network, star, comb, or ladder types of polymer. The polymer can be a homopolymer in which a single monomer is used or can be copolymer in which two or more monomers are used. Types of copolymers include alternating, random, block and graft.

The main chain of a polymer is composed of the atoms whose bonds are required for propagation of polymer length. The side chain of a polymer is composed of the atoms whose bonds are not required for propagation of polymer length.

To those skilled in the art of polymerization, there are several categories of polymerization processes that can be utilized in the described process. The polymerization can be chain or step. This classification description is more often used that the previous terminology of addition and condensation polymer. “Most step-reaction polymerizations are condensation processes and most chain-reaction polymerizations are addition processes” (M. P. Stevens Polymer Chemistry: An Introduction New York Oxford University Press 1990). Template polymerization can be used to form polymers from daughter polymers.

Step Polymerization—In step polymerization, the polymerization occurs in a stepwise fashion. Polymer growth occurs by reaction between monomers, oligomers and polymers. No initiator is needed since there is the same reaction throughout and there is no termination step so that the end groups are still reactive. The polymerization rate decreases as the functional groups are consumed.

Typically, step polymerization is done either of two different ways. One way, the monomer has both reactive functional groups (A and B) in the same molecule so that

A-B yields-[A-B]-

Or the other approach is to have two difunctional monomers.

A-A+B-B yields-[A-A-B-B]-

Yet another approach is to have one difunctional monomer so that

A-A plus another agent yields-[A-A]-.

Chain Polymerization—In chain-reaction polymerization growth of the polymer occurs by successive addition of monomer units to limited number of growing chains. The initiation and propagation mechanisms are different and there is usually a chain-terminating step. The polymerization rate remains constant until the monomer is depleted.

Other Components of the Monomers and Polymers—The polymers have other groups that increase their utility. These groups can be incorporated into monomers prior to polymer formation or attached to the polymer after its formation. These groups include: Targeting Groups—such groups are used for targeting the polymer-nucleic acid complexes to specific cells or tissues. Examples of such targeting agents include agents that target to the asialoglycoprotein receptor by using asialoglycoproteins or galactose residues. Other proteins such as insulin, EGF, or transferrin can be used for targeting. Protein refers to a molecule made up of 2 or more amino acid residues connected one to another as in a polypeptide. The amino acids may be naturally occurring or synthetic. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as fatty acids, cholesterol, dansyl compounds, and amphotericin derivatives.

After interaction of the supramolecular complexes with the cell, other targeting groups can be used to increase the delivery of the drug or nucleic acid to certain parts of the cell. For example, agents can be used to disrupt endosomes and a nuclear localizing signal (NLS) can be used to target the nucleus.

A variety of ligands have been used to target drugs and genes to cells and to specific cellular receptors. The ligand may seek a target within the cell membrane, on the cell membrane or near a cell. Binding of ligands to receptors typically initiates endocytosis. Ligands could also be used for DNA delivery that bind to receptors that are not endocytosed. For example peptides containing RGD peptide sequence that bind integrin receptor could be used. In addition viral proteins could be used to bind the complex to cells. Lipids and steroids could be used to directly insert a complex into cellular membranes.

The polymers can also contain cleavable groups within themselves. When attached to the targeting group, cleavage leads to reduce interaction between the complex and the receptor for the targeting group. Cleavable groups include but are not restricted to disulfide bonds, diols, diazo bonds, ester bonds, sulfone bonds, acetals, ketals, enol ethers, enol esters, enamines and imines.

Polyelectrolyte—A polyelectrolyte, or polyion, is a polymer possessing more than one charge, i.e. the polymer contains groups that have either gained or lost one or more electrons. A polycation is a polyelectrolyte possessing net positive charge, for example poly-L-lysine hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polyelectrolyte containing a net negative charge. The polyanion can contain monomer units that are charge negative, charge neutral, or charge positive, however, the net charge on the polymer must be negative. A polyanion can also mean a non-polymeric molecule that contains two or more negative charges. The term polyelectrolyte includes polycation, polyanion, zwitterionic polymers, and neutral polymers. The term zwitterionic refers to the product (salt) of the reaction between an acidic group and a basic group that are part of the same molecule.

Steric Stabilizer—A steric stabilizer is a long chain hydrophilic group that prevents aggregation by sterically hindering particle to particle or polymer to polymer electrostatic interactions. Examples include: alkyl groups, PEG chains, polysaccharides, alkyl amines. Electrostatic interactions are the non-covalent association of two or more substances due to attractive forces between positive and negative charges.

Buffers—Buffers are made from a weak acid or weak base and their salts. Buffer solutions resist changes in pH when additional acid or base is added to the solution.

Biological, Chemical, or Biochemical reactions—Biological, chemical, or biochemical reactions involve the formation or cleavage of ionic and/or covalent bonds.

Reactive—A compound is reactive if it is capable of forming either an ionic or a covalent bond with another compound. The portions of reactive compounds that are capable of forming covalent bonds are referred to as reactive functional groups or reactive groups.

Steroid—A steroid derivative means a sterol, a sterol in which the hydroxyl moiety has been modified (for example, acylated), a steroid hormone, or an analog thereof. The modification can include spacer groups, linkers, or reactive groups.

Sterics—Steric hindrance, or sterics, is the prevention or retardation of a chemical reaction because of neighboring groups on the same molecule.

Lipid—Any of a diverse group of organic compounds that are insoluble in water, but soluble in organic solvents such as chloroform and benzene. Lipids contain both hydrophobic and hydrophilic sections. The term lipids is meant to include complex lipids, simple lipids, and synthetic lipids.

Complex Lipids—Complex lipids are the esters of fatty acids and include glycerides (fats and oils), glycolipids, phospholipids, and waxes.

Simple Lipids—Simple lipids include steroids and terpenes.

Synthetic Lipids—Synthetic lipids includes amides prepared from fatty acids wherein the carboxylic acid has been converted to the amide, synthetic variants of complex lipids in which one or more oxygen atoms has been substituted by another heteroatom (such as Nitrogen or Sulfur), and derivatives of simple lipids in which additional hydrophilic groups have been chemically attached. Synthetic lipids may contain one or more labile groups.

Fats—Fats are glycerol esters of long-chain carboxylic acids. Hydrolysis of fats yields glycerol and a carboxylic acid—a fatty acid. Fatty acids may be saturated or unsaturated (contain one or more double bonds).

Oils—Oils are esters of carboxylic acids or are glycerides of fatty acids.

Glycolipids—Glycolipids are sugar containing lipids. The sugars are typically galactose, glucose or inositol.

Phospholipids—Phospholipids are lipids having both a phosphate group and one or more fatty acids (as esters of the fatty acid). The phosphate group may be bound to one or more additional organic groups.

Wax—Waxes are any of various solid or semisolid substances generally being esters of fatty acids.

Fatty Acids—Fatty acids are considered the hydrolysis product of lipids (fats, waxes, and phosphoglycerides).

Surfactant—A surfactant is a surface active agent, such as a detergent or a lipid, which is added to a liquid to increase its spreading or wetting properties by reducing its surface tension. A surfactant refers to a compound that contains a polar group (hydrophilic) and a non-polar (hydrophobic) group on the same molecule. A cleavable surfactant is a surfactant in which the polar group may be separated from the nonpolar group by the breakage or cleavage of a chemical bond located between the two groups, or to a surfactant in which the polar or non-polar group or both may be chemically modified such that the detergent properties of the surfactant are destroyed.

Detergent—Detergents are compounds that are soluble in water and cause nonpolar substances to go into solution in water. Detergents have both hydrophobic and hydrophilic groups

Micelle—Micelles are microscopic vesicles that contain amphipathic molecules but do not contain an aqueous volume that is entirely enclosed by a membrane. In micelles the hydrophilic part of the amphipathic compound is on the outside (on the surface of the vesicle). In inverse micelles the hydrophobic part of the amphipathic compound is on the outside. The inverse micelles thus contain a polar core that can solubilize both water and macromolecules within the inverse micelle.

Liposome—Liposomes are microscopic vesicles that contain amphipathic molecules and contain an aqueous volume that is entirely enclosed by a membrane.

Microemulsions—Microemulsions are isotropic, thermodynamically stable solutions in which substantial amounts of two immiscible liquids (water and oil) are brought into a single phase due to a surfactant or mixture of surfactants. The spontaneously formed colloidal particles are globular droplets of the minor solvent, surrounded by a monolayer of surfactant molecules. The spontaneous curvature, HO of the surfactant monolayer at the oil/water interface dictates the phase behavior and microstructure of the vesicle. Hydrophilic surfactants produce oil in water (O/W) microemulsions (H0>0), whereas lipophilic surfactants produce water in oil (W/O) microemulsions.

Hydrophobic Groups—Hydrophobic groups indicate in qualitative terms that the chemical moiety is water-avoiding. Typically, such chemical groups are not water soluble, and tend not to form hydrogen bonds.

Hydrophilic Groups—Hydrophilic groups indicate in qualitative terms that the chemical moiety is water-preferring. Typically, such chemical groups are water soluble, and are hydrogen bond donors or acceptors with water.

### EXAMPLES

### Example 1

Preparation of Dodecylamine Hydrochloride:

Dodecylamine (113 mg, 0.610 mmol, Aldrich Chemical Company) was converted to its hydrochloride salt by dissolving in methanol (500 μL), and adding hydrogen chloride (7 mL, 1 M in diethyl ether, Aldrich). The resulting precipitate was washed with diethyl ether and dried under vacuum to afford dodecylamine hydrochloride (115 mg, 85% yield) as a white solid.

### Example 2

Preparation of a Complex with pDNA and Dodecylamine Hydrochloride:

To water (50 μL) was added a solution of pDNA (50 μL of 2 mg/mL solution in water, 100 μg, 0.30 μmol in phosphate) with mixing. A solution of dodecylamine hydrochloride in water (13.4 μL, 134 μg, 0.60 μmol) was added with mixing. After 10 min, the precipitate was spun down with centrifugation, and the pellet was washed with water (2×100 μL). The resulting pellet was dried on a lyophilizer over P₂O₅ for 3 days.

### Example 3

Preparation of a Complex with pDNA and Cetyltrimethylammonium Bromide (CTAB):

To water (50 μL) was added a solution of pDNA (50 μL of 2 mg/mL solution in water, 100 μg, 0.30 μmol in phosphate) with mixing. A solution of dodecylamine hydrochloride in water (13.4 μL, 134 μg, 0.60 μmol) was added with mixing. After 10 min, the precipitate was spun down with centrifugation, and the pellet was washed with water (2×100 μL). The resulting pellet lyophilized over P₂O₅ for 3 days.

### Example 4

Preparation of Cy3-DNA-NC₁₂:

To H₂O (15 μL) was added Cy3-DNA (100 μg, 85 μL of 1.17 μg/μL H₂O, 0.30 μmol phosphate) and gently mixed. NC₁₂ (134 μg, 1 3.4 μL of 10 μg/μL H₂O, 0.61 μmol) was added and gently mixed. A pink precipitate formed. The reaction sat at RT for 10 min and was spun down by centrifugation to form a pellet. The supernatant was decanted and the pellet was washed 2× with water. The pellet was lyophilized over P₂O₅ for several days.

### Example 5

Preparation of Oleic Acid Modified Chitosan:

Oleic acid (4.15 μL, 13.2 mmol, Aldrich) was taken up in DMF (342 μL). To the resulting solution was added sulfo-N-hydroxysuccinimide (29 μL of a 100 mg/mL solution in water, 13.2 μmol, Pierce Chemical Company), and 1-(3-dimethylaminopropyl)-3-ethylcarbodiimide hydrochloride (125 μL of a 20 mg/mL solution in DMF, 13.2 μmol, Aldrich) and the resulting solution was stirred at room temperature. After 1 h, the solution was added to chitosan (reagent dissolved in 1% HCl in water to a final concentration of 10 mg/mL, 500 μL of concentrate was dissolved in 500 μL water and adjusted to pH 6.5 with 1 N NaOH, 5.0 mg polymer (85% deacylated), 31 μmol in amine, Fluka Chemical Company) in DMF (3.5 mL). The resulting solution was stirred at room temperature for 16 h to afford oleic acid modified chitosan as a 1 mg/ml solution in 80% DMF/water.

### Example 6

Preparation of Oleic Acid and Lactobionic Acid Modified Chitosan:

A stock solution of lactobionic acid in DMF was prepared to a final concentration of 100 mg/mL. To 9.45 μL of the lactobionic acid stock solution (945 μg, 2.64 μmol, Aldrich) was added DMF (437 μL), sulfo-N-hydroxysuccinimide (28.7 μL of a 20 mg/mL solution in water, 573 μg, 2.64 μmol, Pierce Chemical Company), and 1-(3-dimethyl-aminopropyl)-3-ethylcarboduimide hydrochloride (25.3 μL of a 20 mg/mL solution in DMF, 505 μg, 2.64 μmol, Aldrich). The resulting solution was stirred for 1 h at ambient temperature and added to a solution of oleic acid modified chitosan (1 mL of a 1 mg/ml solution in 80% DMF/water). The resulting solution was stirred for 16 h at ambient temperature to afford oleic acid and lactobionic acid modified chitosan as a 0.667 mg/ml solution.

### Example 7

Preparation of Deoxycholic Acid Modified Chitosan:

Deoxycholic acid (CalBiochem) was dissolved in water to a final concentration of 100 mg/mL. To a solution of the deoxycholic acid in water (11 μL, 2.7 μmol) was added DMF (407 μL), followed by sulfo-N-hydroxysuccinimide (57 μL of a 10 mg/mL solution in water, 2.7 μmol, Pierce Chemical Company), and 1-(3-dimethylaminopropyl)-3-ethylcarbodiimide hydrochloride (25 μL of a 20 mg/mL solution in DMF, Aldrich) and the resulting solution was stirred at RT. After 1 h, chitosan (reagent dissolved in 1% HCl in water to a final concentration of 10 mg/mL, 1 mL of concentrate was dissolved in 5 mL water and adjusted to pH 6.5 with 3 N NaOH, 10 mg polymer (85% deacylated), 53 μmol in amine, Fluka Chemical Company) was added and the resulting solution was stirred for 16 h at room temperature to afford the deoxycholic acid modified chitosan.

### Example 8

Preparation of Cholic Acid Modified Chitosan:

Cholic acid (CalBiochem) was dissolved in water to a final concentration of 100 mg/mL. To a solution of the cholic acid in water (11 μL, 2.7 μmol) was added DMF (407 μL), followed by sulfo-N-hydroxysuccinimide (57 μL of a 10 mg/mL solution in water, 2.7 μmol, Pierce Chemical Company), and 1-(3-dimethylaminopropyl)-3-ethylcarboduimide hydrochloride (25 μL of a 20 mg/mL solution in DMF, Aldrich) and the resulting solution was stirred at RT. After 1 h, chitosan (reagent dissolved in 1% HCl in water to a final concentration of 10 mg/mL, 1 mL of concentrate was dissolved in 5 mL water and adjusted to pH 6.5 with 3 N NaOH, 10 mg polymer (85% deacylated), 53 μmol in amine, Fluka Chemical Company) was added and the resulting solution was stirred for 16 h at room temperature to afford the cholic acid modified chitosan.

### Example 9

Preparation of 2-Propionic-3-methylmaleic anhydride (carboxydimethylmaleic anhydride or CDM):

To a suspension of sodium hydride (0.58 g, 25 mmol) in 50 mL anhydrous tetrahydrofuran was added triethyl-2-phosphonopropionate (7.1 g, 30 mmol). After bubbling of hydrogen gas stopped, dimethyl-2-oxoglutarate (3.5 g, 20 mmol) in 10 mL anhydrous tetrahydrofuran was added and stirred for 30 minutes. Water, 10 mL, was then added and the tetrahydrofuran was removed by rotary evaporation. The resulting solid and water mixture was extracted with 3×50 mL ethyl ether. The ether extractions were combined, dried with magnesium sulfate, and concentrated to a light yellow oil. The oil was purified by silica gel chromatography elution with 2:1 ether:hexane to yield 4 gm (82% yield) of pure triester. The 2-propionic-3-methylmaleic anhydride then formed by dissolving of this triester into 50 mL of a 50/50 mixture of water and ethanol containing 4.5 g (5 equivalents) of potassium hydroxide. This solution was heated to reflux for 1 hour. The ethanol was then removed by rotary evaporation and the solution was acidified to pH 2 with hydrochloric acid. This aqueous solution was then extracted with 200 mL ethyl acetate, which was isolated, dried with magnesium sulfate, and concentrated to a white solid. This solid was then recrystallized from dichloromethane and hexane to yield 2 g (80% yield) of 2-propionic-3-methylmaleic anhydride.

### Example 10

Preparation of 2-(Dodecylpropionamide)-3-methylmaleic anhydride (CDMNC₁₂):

2-Propionic-3-methylmaleic anhydride (100 mg, 0.543 mmol) was dissolved in dichloromethane (3 mL). The resulting solution was cooled to 0° C. in an ice bath, and oxalyl chloride (49.7 μL, 0.57 mmol, Aldrich) was added dropwise by syringe. The resulting solution was allowed to warm to room temperature, and dodecylamine (206 mg, 1.11 mmol, Aldrich) was added, followed by diisopropylethylamine (94.6 μL, 0.543 mmol, Aldrich). After 16 h, the solution was concentrated under reduced pressure (aspirator), and partitioned in ethyl acetate and water. The organic layer was washed with IN HCl (3×), brine, dried (Na₂SO₄), filtered, and concentrated to afford 179 mg (94%) of 2-(dodecylpropionamide)-3-methylmaleic anhydride (CDMNC₁₂). M+1=352.2

### Example 11

Preparation of Chitosan-CDMC₁₂ (Chit-CDMC₁₂):

To Chitosan (200 μg, 20 μL of 10 μg/μL 1% HCl, 1.06 tmol amine, Fluka Chemical Company) was added EtOH (20 μL) and several mg's of diisopropylaminomethyl-polystyrene solid support base (Fluka Chemical Company). CDMC₁₂ (185 μg, 1.85 μL of 100 μg/μL DMF, 0.53 μmol) was added and the reaction was stirred at RT for 30 min, and the solid support base was removed by centrifugation.

### Example 12

Preparation of Chitosan-CDMC12-CDM (Chit-CDMC12-CDM):

To Chit-CDMC12 (200 μg, 100 μL of 2 μg/μL EtOH, 0.53 μmol amine) was added several mg's of diisopropylaminomethyl-polystyrene solid support base (Fluka Chemical Company). 2-Propionic-3-methylmaleic anhydride (CDM) (97 μg, 10 μL of 10 μg/μL DMF, 0.53 μmol) was added and the reaction was stirred at RT for 30 min, and the solid support base was removed by centrifugation.

### Example 13

Preparation of Chitosan-Oleic-CDM (Chit-Ol-CDM):

To Chit-Ol (500 μg, 100 μL of 5 μg/μL 80% DMF, 1.3 μmol) was added several mg's of diisopropyl-aminomethyl-polystyrene solid support base (Fluka Chemical Company) and mixed well. CDM (244 μg, 2.4 μL of 100 μg/μL DMF, 1.3 μmol) was added, the reaction was stirred at RT for 30 min, and the solid support base was removed by centrifugation.

### Example 14

Preparation of Chitosan-Oleic-Succinic Anhydride (Chit-Ol-SA):

To Chit-Ol (500 μg, 100 μL of 5 μg/μL 80% DMF, 1.3 μmol) was added several mg's of dilsopropylaminomethyl-polystyrene solid support base (Fluka Chemical Company) and mixed well. Succinic anhydride (132 μg, 1.3 μL of 100 μg/μL DMF, 1.3 μmol) was added, the reaction was stirred at RT for 30 min, and the solid support base was removed by centrifugation.

### Example 15

Preparation of MC791-CDM:

MC 791 is a polymer prepared from the polymerization of vinyl ethers. The polymerization feed ratios (molar ratios) for MC791 are: 47% ethyl vinyl ether, 3% octadecyl vinyl ether, and 50% 1-aminoethyl vinyl ether (protected during the polymerization reaction as the phthalimide derivative. To MC791 (200 μg, 20 μL of 10 μg/μL H₂O, 3.2 μmol amine) was added EtOH (80 μL) and a few mg's of diisopropylaminomethyl-polystyrene solid support base (Fluka Chemical Company). The solution was mixed well. CDM (200 μg, 2.0 μL of 100 μg/μL DMF, 1.1 μmol) was added and the reaction was stirred at RT for 30 min. The solid support base was removed by centrifugation.

### Example 16

Preparation of MC791-CDMC12-CDM:

MC 791 is a polymer prepared from the polymerization of vinyl ethers. The polymerization feed ratios (molar ratios) for MC791 are: 47% ethyl vinyl ether, 3% octadecyl vinyl ether, and 50% 1-aminoethyl vinyl ether (protected during the polymerization reaction as the phthalimide derivative. To MC791 (200 μg, 20 μL of 10 μg/μL H₂O, 3.2 μmol amine) was added EtOH (80 μL) and a few mg's of diisopropylaminomethyl-polystyrene solid support base (Fluka Chemical Company). The solution was mixed well. CDMC12 (200 μg, 2.0 μL of 100 μg/μL DMF, 0.57 μmol) was added and the reaction was stirred at RT for 30 min. CDM (200 μg, 2.0 μL of 100 μg/μL DMF, 1.1 μmol) was added and the reaction was stirred at RT for an additional 30 min. The solid support base was removed by centrifugation.

### Example 17

Preparation of MC787-CDM:

MC 787 is a polymer prepared from the polymerization of vinyl ethers. The polymerization feed ratios (molar ratios) for MC791 are: 47% n-propyl vinyl ether, 3% octadecyl vinyl ether, and 50% 1-aminoethyl vinyl ether (protected during the polymerization reaction as the phthalimide derivative. To MC787 (200 μg, 20 μL of 10 μg/μL H₂O, 3.2 μmol amine) was added EtOH (80 μL) and a few mg's of diisopropylaminomethyl-polystyrene solid support base (Fluka Chemical Company). The solution was mixed well. CDM (200 μg, 2.0 μL of 100 μg/μL DMF, 1.1 μmol) was added and the reaction was stirred at RT for 30 min. The solid support base was removed by centrifugation.

### Example 18

Preparation of MC787-CDMC12-CDM:

MC 787 is a polymer prepared from the polymerization of vinyl ethers. The polymerization feed ratios (molar ratios) for MC791 are: 47% n-propyl vinyl ether, 3% octadecyl vinyl ether, and 50% 1-aminoethyl vinyl ether (protected during the polymerization reaction as the phthalimide derivative. To MC787 (200 μg, 20 μL of 10 μg/μL H₂O, 3.2 μmol amine) was added EtOH (80 μL) and a few mg's of diisopropylaminomethyl-polystyrene solid support base (Fluka Chemical Company). The solution was mixed well. CDMC12 (200 μg, 2.0 μL of 100 μg/μL DMF, 0.57 μmol) was added and the reaction was stirred at RT for 30 min. CDM (200 μg, 2.0 μL of 100 μg/μL DMF, 1.1 μmol) was added and the reaction was stirred at RT for an additional 30 min. The solid support base was removed by centrifugation.

### Example 19

Condensation of pDNA with Cationic Surfactants.

Part A: Determination of Rhodamine Labeled DNA Condensation with Cetyltrimethyl-ammonium Bromide (CTAB):

PCILuc DNA (pDNA) [Zhang et al. 1997] was modified to a level of 1 rhodamine per 100 bases using Mirus' Label IT® Rhodamine kit (Rhodamine Containing DNA Labeling Reagent, Mirus Corporation). The modified pDNA (10 μg) was mixed with pDNA that was not labeled (90 μg) in water and diluted to a final concentration of 1 μg/μL with water. The modified pDNA/pDNA mixture (5 μg) was diluted with water to a final volume of 500 μL. Different amounts of CTAB were added to the solution and the fluorescence intensity of the solution was measured using a Cary Eclipse Fluorescence Spectrophotometer (ex=555, em=585, Varian, Inc.). Trubetskoy et al have shown that rhodamine labeled pDNA undergoes fluorescence quenching as the pDNA is condensed [Trubetskoy, 1999].

| N/P | μg CTAB | mM CTAB | Fluor Int |
|---|---|---|---|
| 0 | 0 | 0.000 | 230.896 |
| 0.4 | 2 | 0.011 | 201.540 |
| 0.7 | 4 | 0.022 | 159.277 |
| 1.1 | 6 | 0.033 | 85.253 |
| 1.4 | 8 | 0.044 | 81.625 |
| 1.8 | 10 | 0.055 | 78.471 |
| 2.2 | 12 | 0.066 | 74.722 |
| 2.5 | 14 | 0.077 | 67.267 |
| 2.9 | 16 | 0.088 | 62.799 |

The results indicate that CTAB condenses the pDNA at a CTAB concentration of 0.33 mM, which is below the literature cmc of CTAB (1 mM)[Calbiochem, 2000-2001].

Part B: Determination of Rhodamine Labeled DNA Condensation with Dodecylamine Hydrochloride:

PCILuc DNA (pDNA) [Zhang et al. 1997] was modified to a level of 1 rhodamine per 100 bases using Mirus' Label It® Rhodamine kit (Rhodamine Containing DNA Labeling Reagent, Mirus Corporation). The modified pDNA (10 μg) was mixed with pDNA that was not labeled (90 μg) in water and diluted to a final concentration of 1 μg/μL with water. The modified pDNA/pDNA mixture (5 μg) was diluted with water to a final volume of 500 μL. Different amounts of dodecylamine hydrochloride were added to the solution and the fluorescence intensity of the solution was measured using a Cary Eclipse Fluorescence Spectrophotometer (ex=555, em=585, Varian, Inc.). Trubetskoy et al have shown that rhodamine labeled pDNA undergoes fluorescence quenching as the pDNA is condensed [Trubetskoy, 1999].

| N/P | μg NC12 | mM NC12 | Fluor Int |
|---|---|---|---|
| 0 | 0 | 0.000 | 264.039 |
| 0.6 | 2 | 0.018 | 183.656 |
| 1.2 | 4 | 0.036 | 106.095 |
| 1.8 | 6 | 0.054 | 95.586 |
| 2.4 | 8 | 0.072 | 86.119 |
| 3.0 | 10 | 0.090 | 77.670 |
| 3.6 | 12 | 0.108 | 68.208 |
| 4.2 | 14 | 0.126 | 62.115 |
| 4.8 | 16 | 0.144 | 60.158 |
| 5.4 | 18 | 0.162 | 53.467 |
| 6.0 | 20 | 0.180 | 46.790 |
| 6.5 | 22 | 0.198 | 43.375 |
| 7.1 | 24 | 0.216 | 45.000 |

The results indicate the pDNA is condensed between 0.036 and 0.054 mM dodecylamine hydrochloride concentration. The literature cmc for dodecylamine hydrochloride is 2.5 mM.

### Example 20

Mouse Tail Vein Injections of pDNA-NC12 (pCI Luc) with Modified Chitosan Polymers:

Fifteen complexes were prepared as follows:

Complex I: pDNA (pCI Luc, 40 μg) in H₂O (2 mL). Ringers (10 mL) was added prior to injection.

Complex II: pDNA-NC₁₂ (pCI Luc, 40 μg, 40 μL of 1 μg/μL EtOH) was added to H₂O (2 mL) and vortexed. Ringers (10 mL) was added prior to injection and mixed well.

Complex III: pDNA-NC₁₂ (pCI Luc, 40 μg, 40 μL of 1 μg/μL EtOH) was added to a solution of Chit-Ol (84 μg, 0.22 μmol) in H₂O (2 mL) and vortexed. Ringers (10 mL) was added prior to injection and mixed well.

Complex IV: pDNA-NC12 (pCI Luc, 40 μg, 40 μL of 1 μg/μL EtOH) was added to a solution of Chit-Ol-LBA (84 μg, 0.22 μmol) in H₂O (2 mL) and vortexed. Ringers (10 mL) was added prior to injection and mixed well.

Complex V: pDNA-NC₁₂ (pCI Luc, 40 μg, 40 μL of 1 μg/μL EtOH) was added to a solution of Chit-CDMC12 (84 μg, 0.22 μmol) in H₂O (2 mL) and vortexed. Ringers (10 mL) was added prior to injection and mixed well.

Complex VI: pDNA-NC₁₂ (pCI Luc, 40 μg, 40 μL of 1 μg/μL EtOH) was added to a solution of Chit-Ol-SA (84 μg, 0.22 μmol) in H2O (2 mL) and vortexed. Ringers (10 mL) was added prior to injection and mixed well.

Complex VII: pDNA-NC₁₂ (pCI Luc, 40 μg, 40 μL of 1 μg/μL EtOH) was added to a solution of Chit-Ol-CDM (84 μg, 0.22 μmol) in H₂O (2 mL) and vortexed. Ringers (10 mL) was added prior to injection and mixed well.

Complex VIII:pDNA-NC₁₂ (pCI Luc, 40 μg, 40 μL of 1 μg/μL EtOH) was added to a solution of Chit-CDM (84 μg, 0.22 μmol) in H₂O (2 mL) and vortexed. Ringers (10 mL) was added prior to injection and mixed well.

Complex IX: pDNA-NC₁₂ (pCI Luc, 40 μg, 40 μL of 1 μg/μL EtOH) was added to a solution of Chit-CDM12-CDM (84 μg, 0.22 μmol) in H₂O (2 mL) and vortexed. Ringers (10 mL) was added prior to injection and mixed well.

Complex X: pDNA-NC₁₂ (pCI Luc, 40 μg, 40 μL of 1 μg/μL EtOH) was added to a solution of Chit-LBA-CDM12 (84 μg, 0.22 μmol) in H₂O (2 mL) and vortexed. Ringers (10 mL) was added prior to injection and mixed well.

Complex XI: pDNA-NC₁₂ (pCI Luc, 40 μg, 40 μL of 1 μg/μL EtOH) was added to a solution of Chit-Chol (84 μg, 0.22 μmol) in H₂O (2 mL) and vortexed. Ringers (10 mL) was added prior to injection and mixed well.

Complex XII: pDNA-NC₁₂ (pCI Luc, 40 μg, 40 μL of 1 μg/μL EtOH) was added to a solution of Chit-Chol-CDM12 (84 μg, 0.22 μmol) in H₂O (2 mL) and vortexed. Ringers (10 mL) was added prior to injection and mixed well.

Complex XIII: pDNA-NC₁₂ (pCI Luc, 40 μg, 40 μL of 1 μg/μL EtOH) was added to a solution of Chit-Chol-CDM12-CDM (84 μg, 0.22 μmol) in H₂O (2 mL) and vortexed. Ringers (10 mL) was added prior to injection and mixed well.

Complex XIV: pDNA-NC₁₂ (pCI Luc, 40 μg, 40 μL of 1 μg/μL EtOH) was added to a solution of Chit (84 μg, 0.22 μmol) in H₂O (2 mL) and vortexed. Ringers (10 mL) was added prior to injection and mixed well.

Complex XV: pDNA(pCI Luc, 40 μg) was added to a solution of Chit (84 μg, 0.22 μmol) in H₂O (2 mL) and vortexed. Ringers (10 mL) was added prior to injection and mixed well.

Tail vein injections of 1.0 mL per 10 g body weight (˜2.5 ml) were preformed on ICR mice (n=2) using a 30 gauge, 0.5 inch needle. Injections were done manually with injection times of 4-5 sec [Zhang et al. 1999; Liu et al. 1999]. One day after injection, the livers were harvested and homogenized in lysis buffer (0.1% Triton X-100, 0.1 M K-phosphate, 1 mM DTT, pH 7.8). Insoluble material was cleared by centrifugation and 10 μl of the cellular extract or extract diluted 10× was analyzed for luciferase activity as previously reported [Wolffetal 1990].

Results: 2.5 mL Injections

| | | | |
|---|---|---|---|
| Complex I: | 11,348,425 | Complex IX: | 206,866 |
| Complex II: | 10,106,551 | Complex X: | 48,860 |
| Complex III: | 17,994,604 | Complex XI: | 84,423 |
| Complex IV: | 13,645,295 | Complex XII: | 61,295 |
| Complex V: | 6,880,568 | Complex XIII: | 451,827 |
| Complex VI: | 87,746 | Complex XIV: | 268,552 |
| Complex VII: | 301,174 | Complex XV: | 984,729 |
| Complex VIII: | 98,434 | | |

Results indicate an increased level of pCI Luc DNA expression in pDNA-NC₁₂/modified Chitosan complexes over pCI Luc DNA/Chitosan complexes. These results also indicate that the pDNA is being released from pDNA-NC₁₂/modified Chitosan complexes, and is accessible for transcription.

### Example 21

Mouse Tail Vein Injections of pDNA-NC12 (pCI Luc) with Modified Chitosan Polymers:

Seven complexes were prepared as follows:

Complex I: pDNA (pCI Luc, 30 μg) in H₂O (750 μL).

Complex II: pDNA-NC₁₂ (pCI Luc, 30 μg, 30 μL of 1 μg/μL EtOH) was added to H₂O (750 μL) and vortexed.

Complex III: pDNA-NC₁₂ (pCI Luc, 30 μg, 30 μL of 1 μg/μL EtOH) was added to a solution of Chit-Ol (63 μg, 0.17 μmol) in H₂O (750 μL) and vortexed.

Complex IV: pDNA-NC₁₂ (pCI Luc, 30 μg, 30 μL of 1 μg/μL EtOH) was added to a solution of Chit-CDM12 (63 μg, 0.17 μmol) in H₂O (750 μL) and vortexed.

Complex V: pDNA-NC₁₂ (pCI Luc, 30 μg, 30 μL of 1 μg/μL EtOH) was added to a solution of Chit-Ol-LBA (63 μg, 0.17 μmol) in H₂O (750 μL) and vortexed.

Complex VI: pDNA-NC₁₂ (pCI Luc, 30 μg, 30 μL of 1 μg/μL EtOH) was added to a solution of Chit (63 μg, 0.17 μmol) in H₂O (750 μL) and vortexed.

Complex VII: pDNA (pCI Luc, 30 μg) was added to a solution of Chit (63 μg, 0.17 μmol) in H₂O (750 μL) and vortexed.

250 μL tail vein injections of 250 μL of the complex were preformed on ICR mice (n=2) using a 30 gauge, 0.5 inch needle, with the total solution injected by hand within 10 seconds. One day after injection, the animal was sacrificed, and a luciferase assay was conducted on the liver, spleen, lung, heart, and kidney. Luciferase expression was determined as previously reported (Wolff et al. 1990.). A Lumat LB 9507 (EG&G Berthold, Bad-Wildbad, Germany) luminometer was used.

Results: 250 μL Injections

| | | | |
|---|---|---|---|
| Complex I: | 266 | Complex V: | 1,094 |
| Complex II: | 75 | Complex VI: | 154 |
| Complex III: | 5,439 | Complex VII: | 207 |
| Complex IV: | 194 | | |

Results indicate an increased level of pCI Luc DNA expression in pDNA-NC₁₂/modified Chitosan complexes over pCI Luc DNA/Chitosan complexes. These results also indicate that the pDNA is being released from pDNA-NC₁₂/modified Chitosan complexes, and is accessible for transcription.

### Example 22

Mouse Tail Vein Injections of Cy3-pDNA-NC₁₂ (pCI Luc) with Modified Chitosan Polymers, Histological Examination:

Three complexes were prepared as follows:

Complex I: Cy3-pDNA-NC₁₂ (pCI Luc, 40 μg, 40 μL of 1 μg/μL EtOH) was added to H₂O (500 μL) and vortexed.

Complex II: Cy3-pDNA-NC₁₂ (pCI Luc, 40 μg, 40 μL of 1 μg/μL EtOH) was added to a solution of Chit-Ol (84 μg, 0.22 μmol) in H₂O (500 μL) and vortexed.

Complex III: Cy3-pDNA-NC₁₂ (pCI Luc, 40 μg, 40 μL of 1 μg/μL EtOH) was added to a solution of Chit-Ol-LBA (84 μg, 0.22 μmol) in H₂O (500 μL) and vortexed.

250 μL tail vein injections of 250 μL of the complex were preformed on ICR mice (n=2) using a 30 gauge, 0.5 inch needle, with the total solution injected by hand within 10 seconds. One day after injection, the animal was sacrificed, and a luciferase assay was conducted on the liver, spleen, lung, heart, and kidney. Luciferase expression was determined as previously reported (Wolff et al. 1990.). A Lumat LB 9507 (EG&G Berthold, Bad-Wildbad, Germany) luminometer was used.

### Example 23

Particle Size of Complexes without pCILuc DNA-NC₁₂:

To a solution of H₂O (500 μL) was added modified polymer (amount indicated below) and vortexed. The size of the complexes was measured on a Zeta Plus Particle Sizer (Brookhaven Instrument Corporation).

| Polymer | Amount (μg) | Size (nm) | Counts (kcps) |
|---|---|---|---|
| Chit-CDM12 | 10.5 | 160 | 312 |
| Chit-CDM12-CDM | 10.5 | 137 | 538 |
| Chit-O1 | 10.5 | offscale | 41 |
| Chit-O1-SA | 10.5 | 141 | 143 |
| Chit-O1-CDM | 10.5 | offscale | 109 |
| Chit-LBA-CDM12 | 10.0 | 2865 | 417 |
| MC791-CDM12 | 10.0 | 359 | 42 |
| MC791-CDM12-CDM | 10.0 | 231 | 58 |
| MC787-CDM12 | 10.0 | 87 | 221 |
| MC787-CDM12-CDM | 10.0 | 13686 | 55 |

The particle size data indicates that particles are present in some of the polymer formulations at this concentration, as polymeric micelles.

### Example 24

Particle Size of Complexes with pCILuc DNA-NC₁₂:

To a solution of H₂O (500 μL) was added modified polymer (amount indicated below) and vortexed. DNA-NC₁₂ (5 μg, 5 μL of 1 μL/μg EtOH, 0.015 μmol phosphate) was added to the solution, vortexed and the size of the complexes was measured on a Zeta Plus Particle Sizer (Brookhaven Instrument Corporation).

| Polymer | Amount (μg) | Size (nm) | Counts (kcps) |
|---|---|---|---|
| Chit-CDM12 | 10.5 | 182 | 325 |
| Chit-CDM12-CDM | 10.5 | 160 | 210 |
| Chit-O1 | 10.5 | 252 | 147 |
| Chit-O1-SA | 10.5 | 152 | 264 |
| Chit-O1-CDM | 10.5 | 875 | 12 |
| Chit-LBA-CDM12 | 10.0 | 258 | 393 |
| MC791-CDM12 | 10.0 | 183 | 344 |
| MC791-CDM12-CDM | 10.0 | 170 | 248 |
| MC787-CDM12 | 10.0 | 293 | 2400 |
| MC787-CDM12-CDM | 10.0 | 160 | 556 |

The particle size data indicates particles are present when both the polymer and the modified DNA are present.

### Example 25

Synthesis of 3-Dimethylaminopropyl-1-dimethyloctadecyl silyl ether.

To a solution of 3-dimethylamino-1propanol (90.0 mg, 0.873 mmol, Aldrich Chemical Company) in 2 mL chloroform was added dimethyloctadecyl chlorosilane (378 mg, 1.09 mmol, Aldrich Chemical Company) and imidazole (74.2 mg, 1.09 mmol, Aldrich Chemical Company). After 16 hrs at ambient temperature, the solution was partitioned in EtOAc/H2O with 10% sodium bicarbinate. The organic layer was washed with water, and brine. The solvent was removed (aspirator) to afford 328 mg (91%) of 3-dimethylaminopropyl-1-dimethyloctadecyl silyl ether as a cream colored solid.

### Example 26

Synthesis of 3-(dimethylaminopropyl)-1,2-dimethyloctadecyl silyl ether.

To a solution of 3-(dimethylamino)-1,2-propanediol (50.0 mg, 0.419 mmol, Aldrich Chemical Company) in 2 mL chloroform was added dimethyloctadecyl chlorosilane (328 mg, 0.944 mmol, Aldrich Chemical Company) and imidazole (68.1 mg, 0.944 mmol, Aldrich Chemical Company). After 16 hrs at ambient temperature, the solution was partitioned in EtOAc/H2O with 10% sodium bicarbinate. The organic layer was washed with water, and brine. The solvent was removed (aspirator) to afford 266 mg (86%) of 3-(dimethylaminopropyl)-1,2-dimethyloctadecyl silyl ether as a white solid.

### Example 27

Synthesis of 1-(3-lauroylaminopropyl), 4-(3-oleoylaminopropyl)piperazine (MC763), 1,4-bis(3-lauroylaminopropyl)piperazine (MC762), and 1,4-bis(3-oleoylaminopropyl)piperazine (MC798).

To a 25 mL flame dried flask was added oleoyl chloride (freshly distilled, 1.0 ml, 3.0 mmol, Aldrich Chemical Company) and lauroyl chloride (0.70 mL, 3.0 mmol, Aldrich Chemical Company) in 15 mL dichloromethane under N₂. The resulting solution was cooled to 0° C. in an ice bath. N,N-Diisopropylethylamine (1.1 ml, 6.1 mmol, Aldrich Chemical Company) was added followed by 1,4-bis(3-aminopropyl)piperazine (0.50 ml, 2.4 mmol, Aldrich Chemical Company). The ice bath was removed and the solution stirred at ambient temperature for 15 hr. The solution was washed twice with 1N NaOH (10 ml), twice with water (10 ml), and concentrated under reduced pressure.

Approximatly 30% of the resulting residue was purified by semi-preparative HPLC on a Beta Basic Cyano column (150 Å, 5 μm, 250×21 mm, Keystone Scientific, Inc.) with acetonitrile/H₂O/trifluoroacetic acid eluent. Three compounds were isolated from the column and verified by mass spectroscopy (Sciex API 150EX).

| | |
|---|---|
| MC763 | 1-(3-lauroylaminopropyl),4-(3-oleoylaminopropyl) piperazine |
| | (MW = 647) |
| MC762 | 1,4-bis(3-lauroylaminopropyl)piperazine (MW = 564) |
| MC798 | 1,4-bis(3-oleoylaminopropyl)piperazine (MW = 729.25) |

### Example 28

Synthesis of 1(3-myristoylaminopropyl), 4-(3-oleoylaminopropyl)piperazine (MC765), 1,4-bis(3-myristoylaminopropyl)piperazine (MC764), and 1,4-bis(3-oleoylaminopropyl)piperazine (MC798).

To a 25 mL flame dried flask was added oleoyl chloride (freshly distilled, 1.0 ml, 3.0 mmol) and myristoyl chloride (0.83 ml, 3.0 mmol, Aldrich Chemical Company) in 15 ml dichloromethane under N₂. The resulting solution was cooled to 0° C. in an ice bath. N,N-Diisopropylethylamine (1.1 ml, 6.1 mmol) was added followed by 1,4-bis(3-aminopropyl)piperazine (0.50 ml, 2.4 mmol). The ice bath was removed and the solution stirred at ambient temperature for 15 hr. The solution was washed twice with 1N NaOH (10 ml), twice with water (10 ml), and concentrated under reduced pressure.

Approximatly 30% of the resulting residue was purified by semi-prerarative HPLC on a Beta Basic Cyano column with acetonitrile/H₂O/trifluoroacetic acid eluent. Three compounds were isolated from the column and verified by mass spectroscopy.

| | |
|---|---|
| MC765 | 1-(3-myristoylaminopropyl),4-(3-oleoylaminopropyl) piperazine |
| | (MW = 674) |
| MC764 | 1,4-bis(3-myristoylaminopropyl)piperazine (MW = 620) |
| MC798 | 1,4-bis(3-oleoylaminopropyl)piperazine (MW = 729.25) |

### Example 29

Synthesis of 1,4-bis(3-decanoylaminopropyl)piperazine MC774.

To a solution of 1,4-bis(3-aminopropyl)piperazine (10 μl, 0.049 mmol, Aldrich Chemical Company) in dichloromethane (1 ml) cooled to 0° C., was added decanoyl chloride (25 μl, 0.12 mmol, Aldrich Chemical Company) and N,N-Diisopropylethylamine (21 μl, 0.12 mmol). After 30 min, the solution was allowed to warm to ambient temperature. After 12 hrs, the solution was washed with water (2×2 ml), and concentrated under reduced pressure to afford 1,4-bis(3-decanoylaminopropyl)piperazine (MC774) (21.6 mg, 87%) of sufficient purity by TLC.

### Example 30

Synthesis of 1,4-bis(3-palmitoylaminopropyl)piperazine (MC775).

To a solution of 1,4-bis(3-aminopropyl)piperazine (10 μl, 0.049 mmol) in dichloromethane (1 mL), was added palmitoleic acid (30.8 mg, 0.12 mmol, Aldrich Chemical Company), N,N-Diisopropylethylamine (21 μL, 0.12 mmol), and dicyclohexylcarbodiimide (25 mg, 0.12 mmol). After 12 hrs, the solution was filtered and washed with water (2×2 mL), and concentrated under reduced pressure to afford 1,4-bis(3-palmitoylaminopropyl)piperazine (MC775) (26.5 mg, 81%) of sufficient purity by TLC.

### Example 31

Synthesis of 1,4-bis(3-thioctoylaminopropyl)piperazine (MC777), 1(3-linolenoylaminopropyl), 4-(3-thioctoylaminopropyl)viperazine (MC778), 1,4-bis(3-linolenoylaminopropyl)piperazine (MC779).

To a solution of benzotriazole-1-yl-oxy-tris-pyrrolidino-phosphonium hexafluorophosphate (PyBOP, 1.300 g, 2.500 mmol, NovaBiochem) in dichloromethane (8 ml) was added thioctic acid (0.248 g, 1.20 mmol, Aldrich Chemical Company) and linolenic acid (365 μl, 1.20 mmol, Aldrich Chemical Company). To the resulting solution was added 1,4-bis(3-aminopropyl)-piperazine (206 μl, 1.00 mmol) followed by N,N-Diisopropylethylamine (610 μl, 3.5 mmol). After 16 hrs at ambient temperature, the solution was washed with water (2×20 ml), and concentrated under reduced pressure to afford 1,800 g of crude material. A 85 mg portion of the crude material was dissolved in 2 ml of acetonitrile (0.1% trifluoroacetic acid)/1 ml of water (0.1% trifluoroacetic acid), and purified by reverse phase HPLC (10-90% B over 40 min) on a Beta Basic Cyano column to afford 31.8 mg MC777, 1.3 mg MC778, and 1.5 mg MC779.

### Example 32

Synthesis of 1,4-bis(3-(trans)-retinoylaminopropyl)piperazine (MC780), 1-(3-(cis-11)-eicosenoylaminopropyl), 4-(3-(trans)-retinoylaminopropyl)piperazine (MC781), 1,4-bis(3-(cis-11)-eicosenoylaminopropyl)piperazine (MC782).

Compounds MC780, MC 781, and MC782 were made using a similar synthesis to compounds MC777, MC778, and MC779. The crude material from the synthesis was dissolved in 2 ml of acetonitrile (0.1% trifluoroacetic acid)/1 ml of water (0.1% trifluoroacetic acid), and purified by reverse phase HPLC (10-90% B over 40 min) on a Beta Basic Cyano column to afford 7.1 mg MC780, 13.0 mg MC781, and 18.0 mg MC782.

### Example 33

Synthesis of bis(2-aminoethyl)-(2-oleoylaminoethyl)amine (MC753), 2-aminoethyl-bis-(2-oleoylaminoethyl)amine (MC754), and tris-(2-oleoylaminoethyl)amine (MC755).

Tris(2-aminoethyl)amine (0.2 mL, 1.4 mmol, Aldrich Chemical Company) as taken up in dichloromethane (3 mL, 0.5 M) and cooled in a dry ice/acetone bath. To the resulting solution was added oleoyl chloride (0.18 mL, 0.45 mmol, Aldrich Chemical Company), dropwise, while under a blanket of N₂. The reaction was warmed to ambient temperature with stirring. The reaction was stirred at rt for 1 hr and analyzed by TLC and mass spectrometry (Sciex API 150EX) to verify that the reaction was complete. The reaction was concentrated under reduced pressure to afford a beige film (330 mg). A portion of the material was purified by reverse phase HPLC on a Diphenyl column (Vydac), 40-90% B over 20 min (A=0.1% TFA/H₂O, B=0.1% TFA/acetonitrile). Four HPLC runs were performed. Fractions were collected at 210 nm and analyzed by mass spectrometry (Sciex API 150EX). Fractions for MC753 (5,6,15-17,25-31,42-44), MC754 (7,18,19,32,33,45,46) and MC755(8-10,20-3,34-7,47-50) were pooled together, concentrated under reduced pressure, froze and lyophilized. Yielded MC753 (26 mg), MC754 (8.6 mg) and MC755 (150 mg).

### Example 34

Synthesis of S-oleoyl-N-acetyl-L-cysteine-3-(dimethylaminopropylamine)-amide (MC909).

Preparation of Di-(Dimethylamino)propylamino-Cystine: To a flame dried round bottom flask (25 mL) was added N,N-Di-Boc-L-Cystine (500 mg, 1.1 mmol, Aldrich Chemical Company) and taken up in THF (6 mL, 0.2 M) with stirring. To the resulting solution was added N-hydroxysuccinimde (260 mg, 2.3 mmol, Aldrich Chemical Company) and dicyclohexylcarbodiimide (520 mg, 2.5 mmol, Aldrich Chemical Company). After 2 min, 3-(Dimethylamino)propylamine (0.28 mL, 230 mg, 1.1 mmol, Aldrich Chemical Company) was added to the reaction mixture. The reaction was stirred at rt for 30 min and analyzed by TLC and mass spectrometry (Sciex API 150EX) to verify that the reaction was complete. The reaction was filtered to remove the DCU and concentrated under reduced pressure. The resulting resudue was precipitated in cold ether (10 mL) and washed with ether (2×10 mL). The precipitate was dried under N₂ then placed on high vacuum to afford di-(dimethylamino)propylamino-di-Boc-cystine as a white powder (670 mg, 97%).

Di-(dimethylamino)propylamino-di-Boc-L-cystine (670 mg, 1.1 mmol) was taken up in TIPS/H₂O/TFA (0.5/4.5/95% vol, 5 mL, 0.2 M) covered with N₂ and stirred at rt for 1 hr. Deprotection of the material was verified by TLC and mass spectrometry (Sciex API 150EX). The material was precipitated in cold ether (10 mL), washed in ether (2×10 mL), dried under N₂ and placed under vacuum to afford di-(dimethylamino)propylamino-cystine as a white powder (670 mg, 100%).

To a solution of di-(dimethylamino)propylamino-cystine (670 mg, 1.6 mmol) in 1-methyl-2-pyrrolidinone (8 mL, 0.2 M, VWR), was added diisopropylethylamine (0.58 mL, 430 mg, 3.3 mmol, Aldrich Chemical Company) with stirring. The reaction was cooled in an ice bath (0° C.) and acetic anhydride (1.5 mL, 1.7 g, 16 mmol, Aldrich Chemical Company) was slowly added. The reaction mixture was warmed to ambient temperature naturally and continued to stir for 1 hr. Amine capping was verified by TLC and mass spectrometry (Sciex API 150EX). The reaction solution was added dropwise to cold ether (25 mL) and a white precipitate formed. The precipitate was washed with ether (2×25 mL), dried under N₂ then placed under vacuum. Yielded a dark yellow oil (200 mg, 25%).

The yellow oil (83 mg, 0.17 mmol) was taken up in THF (2 mL, 0.2 M) with stirring. Dithiolthreitol (35 mg, 0.23 mmol, 0.1 M, Sigma Chemical Company) was added with stirring. The reaction stirred at rt for 16 hr. The material was analyzed by TLC and mass spectrometry (Sciex API 150EX) to verify that the disulfide cleavage was complete. A portion of the material (54 mg) was purified by reverse phase HPLC on an Aquasil C18 column (Keystone Scientific Inc.), 10-40% B, 0-10 min, 4-60% B 10-20 min, 60-90% B 20-30 min (A=0.1% TFA in H₂O, B=0.1% TFA in acetonitrile). Fractions were collected at 210 nm and analyzed by mass spectrometry (Sciex API 150EX). Fractions (3, 4 30, 31) that contained desired product from two HPLC runs were pooled together, concentrated under reduced pressure, froze and lyophilized. Afforded 32 mg (60%) as a clear oil. The oil (32 mg, 0.13 mmol) was taken up with THF (1 mL, 0.13 M) with stirring. To the resulting solution was added diisopropylethylamine (0.02 mL, 17 mg, 0.13 mmol, Aldrich Chemical Company). The reaction solution was cooled in an ice bath (0° C.) and oleoyl chloride (39 mg, 0.13 mmol, Aldrich Chemical Company) was added dropwise by syringe with stirring. The reaction was allowed to warm to ambient temperature naturally. The reaction was analyzed by TLC and mass spectrometry (Sciex API 150EX) to verify that the reaction was complete. The reaction solution was precipitated in cold ether (5 mL), washed with ether (2×5 mL), dried under N₂ and placed on high vacuum. TLC and mass spectrometry (Sciex API 150EX) showed sufficient purity. Yielded S-oleoyl-N-acetyl-L-cysteine-3-(dimethylaminopropylamine)-amide (MC909) as a yellow oil (45.3 mg, 68%).

### Example 35

Preparation of S-oleoyl, N-acetvyl 3-(dimethylaminopropylamine)-L-cysteine-amide (MC909) Hydrochloride.

MC909 (9 mg, 0.018 mmol) was taken up in ethanol (0.09 mL) and added dropwise to cold HCl/ether (1 mL of 1N, Aldrich Chemical Company). The precipitate was spun down by centrifugation, washed with ether (2×1 mL), dried under N₂ and placed on high vacuum. Yielded white crystalline material (6 mg, 62%).

### Example 36

Synthesis of β-D-Glucopyranosyl Decane Disulfide and O-Glycine-β-D-Glucopyranosyl Decane Disulfide. (MC749).

To a solution of decanethiol (0.59 mL, 2.9 mmol, Aldrich Chemical Company) chloroform (11 mL) was added sulfuryl chloride (0.46 mL, 5.7 mmol, Aldrich Chemical Company), and the resulting mixture was stirred at room temperature for 18 hr. Removal of solvent (aspirator), afforded decansulfenyl chloride.

To a solution of decansulfenyl chloride (190 mg, 0.92 mmol) in 4 mL acetonitrile was added 1-thio-β-D-glucose sodium salt hydrate (200 mg, 0.92 mmol, Aldrich Chemical Company) and 15-crown-5 (0.18 mL, 0.899 mmol, Aldrich Chemical Company). The resulting mixture was stirred at ambient temperature for 16 hr, filtered, and precipitated in Et₂O. The residue was triturated with Et₂O and purified by reverse phase HPLC on an Aquasil C18 column (Keystone Scientific Inc.), 10-90% B, 20 min (A=0.1% TFA in H₂O, B=0.1% TFA in Acetonitrile). Lyophilization afforded 10 mg (3%) of β-D-glucopyranosyl decane disulfide as a fine white solid.

To a solution of β-D-glucopyranosyl decane disulfide (8 mg, 0.02 mmol) in 80 μL THF was added N-Boc glycine (15 mg, 0.09 mmol, Sigma Chemical Company), DCC (18 mg, 0.09 mmol, Aldrich Chemical Company), and a catalytic amount of dimethylaminopyridine (Aldrich Chemical Company). The resulting solution was stirred at ambient temperature for 12 hr, and centrifugated to remove the solid. The resulting solution was concentrated under reduced pressure, resuspended in dichloromethane, filtered through a plug of silica gel, and concentrated (aspirator). The Boc protecting group was removed by taking the residue up in 200 μL of 2.5% TIPS/50% TFA/dichloromethane for 12 hr. Removal of solvent (aspirator), followed by purification by reverse phase HPLC on a Aquasil C18 column (Keystone Scientific Inc.), 10-90% B, 20 min (A=0.1% TFA in H₂O, B=0.1% TFA in Acetonitrile) afforded 0.7 mg (5%) of O-glycine-β-D-glucopyranosyl decane disulfide (MC749) as a fine white solid following lyophilization.

### Example 37

Synthesis of β-D-Glucopyranosyl Cholesterol Disulfide.

By similar methodology as described in example 36, β-D-glucopyranosyl cholesterol disulfide was isolated (12% yield).

### Example 38

Synthesis of Two Tailed β-D-Glucopyranosyl Disulfide Derivatives. β-D-Glucopyranosyl N-Dodecanoyl-Cysteine-Dodecanoate Disulfide and O-Glycine-β-D-Glucopyranosyl N-Dodecanoyl-Cysteine-Dodecanoate Disulfide.

To a solution of N-FMOC-S-Trt-Cysteine (585 mg, 1.0 mmol, NovaBioChem) in dichloromethane (4mL) was added 1-dodecanol (240 mg, 1.3 mmol, Aldrich Chemical Company), DCC (260 mg, 1.3 mmol, Aldrich Chemical Company), and a catalytic amount of dimethylaminopyridine (Aldrich Chemical Company). The resulting solution was stirred at ambient temperature for 30 min, filtered, and purified by flash chromatography on silica gel (10-20% EtOAc/hexane eluent). Removal of solvent (aspirator) afforded 572 mg (76%) of the protected cysteine-dodecanoate.

To a solution of protected cysteine-dodecanoate (572 mg, 0.76 mmol) was added 3 mL of 20% piperidine in DMF. The resulting solution was stirred at ambient temperature for 1 hr, and partitioned in EtOAc/H2O. The aqueous layer was extracted 2×EtOAc. The combined organic layer was washed 2×1N HCl, dried (Na₂SO₄), and concentrated to afford S-Trt-cysteine-dodecanoate. The residue was suspended in dichloromethane (2 mL), and cooled to −20° C. Diisopropylethylamine (0.16 mL, 0.92 mmol, Aldrich Chemical Company) was added followed dodecanoyl chloride (0.26 mL, 1.1 mmol, Aldrich Chemical Company), and the solution was allowed to slowly warm to ambient temperature. After 1 hr, the solvent was removed (aspirator), and the residue partitioned in EtOAc/H₂O. The organic layer was washed 2×1 N HCl, 1× brine, dried (Na₂SO₄), and the solvent was removed (aspirator). The resulting residue was suspended in 2% TIPS/50% TFA/dichloromethane to remove the trityl protecting group. After 4 hr the solution was concentrated, and the resulting residue was purified by flash column chromatography on silica gel (10-20% EtOAc/hexanes eluent) to afford 180 mg (42%) N-dodecanoyl-cysteine-dodecanoate (M+1=472.6).

To a solution of N-dodecanoyl-cysteine-dodecanoate (180 mg, 0.38 mmol) in 0.5 mL chloroform was added sulfuryl chloride (62 μL, 0.76 mmol, Aldrich Chemical Company). The resulting solution was stirred at ambient temperature for 2 hr and the solvent was removed (aspirator). The resulting residue was suspended in 1 mL acetonitrile, and 1-thio-β-D-glucose sodium salt hydrate (85 mg, 0.39 mmol, Aldrich Chemical Company) and 15-crown-5 (76 μL, 0.38 mmol, Aldrich Chemical Company) were added. After 1 hr at ambient temperature the solvent was removed (aspirator) and the residue was partitioned in EtOAc/H2O. The organic layer was concentrated and the resulting residue was purified by flash column chromatography on silica gel (5-10% MeOH/0.1% TFA/dichloromethane eluent) to afford 19 mg (8%) β-D-glucopyranosyl N-dodecanoyl-cysteine-dodecanoate disulfide.

To a solution of β-D-glucopyranosyl N-dodecanoyl-cysteine-dodecanoate disulfide (3.9 mg, 0.0045 mmol) in 100 μL dichloromethane was added N-Boc glycine (3.2 mg, 0.018 mmol, Sigma Chemical Company), DCC (3.8 mg, 0.018 mmol, Aldrich Chemical Company), and a catalytic amount of dimethylaminopyridine (Aldrich Chemical Company). The resulting solution was stirred at ambient temperature for 4 hr, and filtered. The Boc protecting group was removed by taking the residue up in 2 mL of 1% TIPS/50% TFA/dichloromethane for 2 hr. Removal of solvent (aspirator), followed by purification by reverse phase HPLC on a Diphenyl column (Vydaq), 20-90% B, 20 min (A=0.1% TFA in H₂O, B=0.1% TFA in Acetonitrile) afforded 3.6 mg (90%) of O-glycine-β-D-glucopyranosyl decane disulfide as a fine white solid following lyophilization.

### Example 39

General Preparation of Peptides.

Peptides were prepaired by standard solid phase peptide synthesis using an ABI433A Peptide Synthesizer (Applied Biosystems), employing FastMoc chemistry. Peptides were sysnthesized on the 0.1 or 1.0 mmol scale. Deprotections and cleavage of the resin were accomplished utilizing standard deprotection techniques. Peptides were purified by reverse phase HPLC to at least a 90% purity level, and verified by mass spectroscopy (Sciex API 150EX).

### Example 40

Synthesis of the Mixed disulfide of H₂N—CRRRRRRRRR—OH (SEQ ID 11) and N-dodecanoyl-cysteine-dodecanoate (MC756).

The peptide H₂N—CRRRRRRRRR—OH (LH168-100) (5.0 mg, 0.0033 mmol) was taken up with isopropyl alcohol (0.5 mL) and trifluoroacetic acid (0.5 mL). To the resulting solution was added aldrithiol (0.79 mg, 0.0036 mmol of 10 mg/mL isopropyl alcohol, Aldrich Chemical Company). The reaction was stirred at rt for 2 hr and analyzed by mass spectrometry to verify (Sciex API EX150) that product formed. The material was precipitated in cold ether (2 mL) and washed with ether (2×2 mL) and dried under N₂. The dried precipitate was taken up with isopropyl alcohol (0.25 mL) and trifluoroacetic acid (0.25 mL). To the resulting solution was added N-dodecanoyl-cysteine-dodecanoate (0.15 mL, 1.5 mg, of 10 mg/mL ethanol, 0.0033 mmol). The reaction was stirred at rt for 2 hr and analyzed by mass spectrometry (Sciex API EX150) to verify that the product formed. The material was precipitated in cold diethyl ether (4 mL) and washed with diethyl ether(2×4 mL). The precipitate was purified by reverse phase HPLC on a Aquasil C18 column (Keystone Scientific Inc), 5-90% B (A=0.1% TFA/H₂O, B=0.1% TFA/acetonitrile) over 20 min. Fractions were collected at 210 nm and analyzed by mass spectrometry (Sciex API EX150). Fractions (9 and 18) were pooled together from the two HPLC runs, concentrated, froze and lyophilized to afforded MC756 (0.5 mg) as white fluffy crystalline material.

### Example 41

Preparation of 1Decanethiochloride.

1-Decanethiol (0.59 mL, 500 mg, 2.9 mmol, Aldrich Chemical Company) was transferred to a flame dried round bottom flask and taken up with chloroform (11 mL, 0.25 M). To the resulting solution was added sulfuryl chloride (0.46 mL, 770 mg, 5.7 mmol, Aldrich Chemical Company) dropwise over 5 min with stirring. The reaction was stirred at rt under N₂ for 16 hr. The reaction mixture was analyzed by TLC to verify that the reaction was complete. The reaction was concentrated under N₂ then placed on the speed vacuum. Material was stored at −20° C. until use.

### Example 42

Trityl Protection of 3-Mercaptopropionic acid.

3-Mercaptopropionic acid (0.41 mL, 500 mg, 4.7 mmol, Aldrich Chemical Company) was transferred to a flame dried round bottom flask and taken up with dichloromethane (18 mL, 0.26 M). To the resulting solution was added diisopropylethylamine (0.82 mL, 610 mg, 4.7 mmol, Aldrich Chemical Company) followed by the addition of trityl chloride (1.4 g, 4.9 mmol, Aldrich Chemical Company). The solution was stirred at rt under N₂ for 16 hr. The reaction mixture was analyzed by TLC and mass spectrometry (Sciex API 150EX) to verify that the reaction was complete. The reaction mixture was concentrated under reduced pressure and yielded white crystalline material. The white crystalline material was slurried in EtOAc (50 mL) and partitioned in H₂O (50 mL). The reaction was neutralized with 0.1 M NaHCO₃. The organic layer was washed with H₂O (2×50 mL) and brine (50 mL). The organic layer was transferred and concentrated under reduced pressure. The crystals were dried on the high vacuum to afford trityl-mercaptopropionic acid (1.7 g) as white crystalline material. The product was verified by mass spectrometry (Sciex API 150EX).

### Example 43

Acylation of 3-(Dimethylamino)propylamine with Trityl-mercaptopropionic acid. Synthesis of 3-thiol-propionic acid (3-(dimethylamino)propylamine)amide.

Trityl-mercaptopropionic acid (300 mg, 0.86 mmol) was taken up in dichloromethane (3.5 mL, 0.25 M). To the resulting solution was added PyBOP (450 mg, 0.86 mmol, Novabiochem) and stirred at rt for 5 min. 3-(Dimethylamino)propylamine (0.11 mL, 0.86 mmol, Aldrich Chemical Company) was added with stirring. The reaction mixture was stirred at rt under N₂ for 16 hr. The reaction mixture was analyzed by TLC and mass spectrometry (Sciex API 150EX) to verify that the reaction was complete. The reaction mixture was concentrated under reduced pressure and taken up in EtOAc (4 mL) and partitioned in H₂O (4 mL), washed with H₂O (2×4 mL) and brine (4 mL). The material was dried over Na₂SO₄, filtered and concentrated under reduced pressure. The concentrated material was taken up with TIPS/TFA/CH₂Cl₂ (2.5/47.5/50% vol, 3 mL) and stirred at rt for 1 hr. The reaction was monitored by TLC and after 1 hr, the deprotection was not complete. TFA (1.5 mL) was added to the solution and after 10 min the solution turned from yellow to clear. The deprotected product was verified by TLC. The reaction was concentrated under reduced pressure and yielded 3-thiol-propionic acid (3-(dimethylamino)propylamine)amide, as a crystalline slurry.

### Example 44

Synthesis of the Mixed disulfide of 3-thiol-propionic acid (3-(dimethylamino)propylamine)amide and decanethiol (MC744).

3-Thiol-propionic acid (3-(dimethylamino)propylamine)amide (82 mg, 0.43 mmol) was taken up in dichloromethane (1.8 mL, 0.25 M). To the resulting solution was added 1-decanethiochloride (90 mg, 0.43 mmol) with stirring. The reaction was stirred at rt under N₂ and instantly turned orange in color. After 20 min, the reaction mixture was analyzed by TCL and mass spectrometry (Sciex API 150EX) to verify that the reaction was complete. The material was stored at −20° C. for 16 hr. Crystals formed (PyBop urea) and were filtered and washed with dichloromethane. The resulting solution was concentrated under reduced pressure and taken up with dichloromethane (1 mL) and purified by flash chromatography (10% MeOH/CH₂Cl₂ eluent) and fractions were collected. The fractions were analyzed by TLC and mass spectrometry (Sciex API 150EX). The desired product was in the final fraction and yielded MC 744 (17 mg).

### Example 45

Preparation of 1-Dodecanethiolchloride.

1-Dodecanethiol (0.59 mL, 500 mg, 2.5 mmol, Aldrich Chemical Company) was transferred to a flame dried round bottom flask and taken up with chloroform (10 mL, 0.25 M). To the resulting solution was added sulfuryl chloride (0.40 mL, 670 mg, 4.9 mmol, Aldrich Chemical Company) dropwise over 5 min with stirring. The reaction was stirred at rt under N₂ for 3 hrs. The reaction mixture was analyzed by TLC to verify that the reaction was complete. The reaction was concentrated under N₂ then on the speed vacuum. Material was stored at −20° C. until use.

### Example 46

Acylation of 3-(Dimethylamino)propylamine with Trityl-mercaptopropionic acid. Synthesis of 3-thiol-propionic acid (3-(dimethylamino)propylamine)amide.

Trityl-mercaptopropionic acid (500 mg, 1.4 mmol) was taken up in dichloromethane (6 mL, 0.25 M). To the resulting solution was added PyBOP (750 mg, 1.4 mmol, Novabiochem) and stirred at rt for 5 min. 3-(Dimethylamino)propylamine (0.18 mL, 1.4 mmol, Aldrich Chemical Company) was added with stirring. The reaction mixture was stirred at rt under N₂ for 16 hr. The reaction mixture was analyzed by TLC and mass spectrometry (Sciex API 150EX) to verify that the reaction was complete. The reaction mixture was concentrated under reduced pressure and taken up in EtOAc (20 mL) and partitioned in H₂O (20 mL), washed with H₂O (2×20 mL) and brine (20 mL). The material was dried over Na₂SO₄, filtered and concentrated under reduced pressure. The concentrated material was taken up in dichloromethane (10 mL) and formed a white precipitate. The material was spun down by centrifugation and the precipitate was washed with dichloromethane (10 mL). The precipitated material was taken up with TIPS/TFA/CH₂Cl₂ (2.5/50/47% vol, 6 mL) and stirred at rt for 1.5 hr. The mixture immediately turned from clear to bright yellow (release of the trityl cation) and back to clear. Deprotection of the material was verified by TLC and mass spectrometry (Sciex API 150EX). The reaction mixture was concentrated under reduced pressure and yielded 3-thiol-propionic acid (3-(dimethylamino)propylamine)amide as a white crystalline solid (245 mg, 91% yield).

### Example 47

Synthesis of Synthesis of the Mixed Disulfide of 3-thiol-propionic acid (3-(dimethylamino)propylamine)amide and dodecanethiol (MC745).

3-Thiol-propionic acid (3-(dimethylamino)propylamine)amide (100 mg, 0.52 mmol) was taken up in dichloromethane (2 mL, 0.25 M). To the resulting solution was added 1dodecanethiochloride (120 mg, 0.52 mmol) with stirring. The reaction was stirred at rt under N₂ and instantly turned orange in color. After 1 hr, the reaction mixture was analyzed by TLC and mass spectrometry (Sciex API 150EX) to verify that the reaction was complete. The resulting solution was concentrated under reduced pressure, yielded a yellow oil (300 mg). The oil (160 mg) was taken up with dichloromethane (0.5 mL) and purified by flash chromatography (10% MeOH/CH₂Cl₂, 0.1% TFA) and fractions were collected. The percentage of MeOH was slowly increased to 20% and fractions were collected. The fractions were analyzed by TLC and mass spectrometry (Sciex API 150EX). The desired product was in the final fraction. The final fraction was concentrated under reduced pressure and yielded MC745 (22.4 mg, 22% yield) as a yellow oil.

### Example 48

Mono Protection of 1,12-Diaminododecane with BOC.

1,12-Diaminododecane (1.0 g, 10 mmol amine, Aldrich Chemical Company) was taken up with dichloromethane (25 mL) and H₂O (25 mL). Sodium hydroxide (2 pellets, Fisher Scientific) were added and the reaction was stirred vigorously. Di-tert-butyl dicarbonate (500 mg, 2.3 mmol, Aldrich Chemical Company) was taken up in dichloromethane (10 mL) and added dropwise to the stirring reaction over 5 min. After 1 hr, the reaction was analyzed by TLC and mass spectrometry (Sciex API 150EX) to verify that the reaction was complete. The reaction was transferred to a separatory funnel and the layers were separated. The organic layer was concentrated under reduced pressure. Half of the concentrated material was brought up in hot acetonitrile (100 mL), and crystallized. The material was spun down by centrifugation and washed with acetonitrile (2×25 mL). The material was dried on high vacuum and yielded crystalline material (745 mg, 99% yield). The product, Boc-aminedodecaneamine was verified by TLC and mass spectrometry (Sciex API 150EX).

### Example 49

Preparation of Trimethylaminododecaneamine.

Boc-aminedodecaneamine (745 mg, 2.5 mmol) was taken up in acetonitrile (12 mL, 0.2 M). To the resulting solution was added diisopropylethylamine (0.43 mL, 370 mg, 2.5 mmol, Aldrich Chemical Company) and methyl iodide (0.77 mL, 1.8 g, 12.4 mmol, Aldrich Chemical Company). The reaction was stirred under N₂ at rt for 16 hr. The reaction was analyzed by TLC and mass spectrometry (Sciex API 150EX) to verify that the reaction was complete. The reaction mixture was heated with a heat gun and allowed to cool to ambient temperature naturally. Crystals formed and were analyzed by TLC and mass spectrometry (Sciex API 150EX). The crystals were determined to be 1,12-dodecaneamine. The crystals were removed by filtration and the supernatant was concentrated under reduced pressure. The concentrated material was taken up with TFA/H₂O/TIPS (95/4.5/1.5% vol, 10 mL) and stirred at rt under N₂. Complete deprotection was verified by TLC. The reaction solution was precipitated in cold ether (25 mL), spun by centrifugation and washed with ether (2×25 mL). The precipitate was dried under N₂ then on high vacuum. Yielded white crystalline material (370 mg). The product was verified by TLC and mass spectrometry (Sciex API 150EX).

### Example 50

Boc Protection of imidazoleacetic acid.

4-Imidazoleacetic hydrochloride (250 mg, 1.5 mmol, Aldrich Chemical Company) was taken up in acetonitrile (5 mL, 0.3 M). To the resulting solution was added triethylamine (0.22 mL, 160 mg, 1.5 mmol, Aldrich Chemical Company). Di-tert-butyl dicarbonate (400 mg, 1.8 mmol, Aldrich Chemical Company) was added followed by a catalytic amount of 4-dimethylaminopyridine (Kodak Chemical Company). After 30 min, the reaction was analyzed by TLC and mass spectrometry (Sciex API 150EX) to verify that the reaction was complete. The reaction mixture was concentrated under reduced pressure, taken up in H₂O (50 mL) and the pH was adjusted to pH 5 with NaOH (1N). The solution was partitioned with EtOAc (50 mL) and washed with brine (2×50 mL). The material was dried over MgSO₄, filtered, concentrated under reduced pressure and yielded an oil (82 mg, 23% yield).

### Example 51

Synthesis of Imidazoleacetic acid (trimethylaminododecaneamine)amide (MC933).

Trimethylaminododecaneamine (53 mg, 0.14 mmol) was taken up in DMF (0.7 mL, 0.2 M). To the resulting solution was added Boc-imidazoleacetic acid (32 mg, 0.14 mmol) followed by dicyclohexylcarbodiimide (30 mg, 0.17 mmol, Aldrich Chemical Company). The reaction was stirred at rt under N₂ for 36 hr. The reaction mixture was cooled to −20° C. and the urea was removed via centrifugation in the form of a pellet. The supernatant was anlalyzed by TLC and mass spectrometry (Sciex API 150EX) to verify that it contained product. The supernatant was concentrated on the speed vacuum and taken up with TIPS/TFA/H₂O (0.1/97.4/2.5% vol, 1.5 mL). The reaction was stirred at rt under N₂ for 1 hr. The reaction was analyzed by TLC and mass spectrometry (Sciex API 150EX) to verify complete deprotection. The reaction mixture was added dropwise to cold ether (10 mL) and formed a white precipitate. The precipitate was washed with ether (2×10 mL), dried under N₂ then placed on high vacuum. Yielded crystalline material (39.2 mg). The material was taken up in acetonitrile (2 mL) with heat. Impurities crystallized and were filtered. The mother liquor was analyzed by TLC and mass spectrometry (Sciex API 150EX) and verified to be product. The mother liquor was concentrated under reduced pressure and afford MC933 as an oil (20 mg, 33% yield).

### Example 52

Preparation of N,N,N-Trimethylaminopropylamine.

N-Boc-1,3-diaminopropane (0.25 mL, 250 mg, 1.4 mmol, Fluka Chemical Company) was taken up with anhydrous acetonitrile (7 mL, 0.2 M). To the resulting solution was added diisopropylethylamine, (0.25 mL, 190 mg, 1.4 mmol Aldrich Chemical Company) followed by methyl iodide (0.31 mL, 720 mg, 5.0 mmol, Aldrich Chemical Company) and the solution was stirred with heat (70C.) under N₂ for 16 hr. The reaction mixture was analyzed by TLC and mass spectrometry (Sciex API 150EX) to verify that the reaction was complete. The reaction mixture was precipitated in cold ether (20 mL), washed with ether (2×20 mL) and dried under N₂. The precipitate was diisopropylethylamine salt (174 mg). The ether layer was concentrated under reduced pressure and taken up in TFA/H₂O/TIPS (97.5/2/0.5% vol. 10 mL). The reaction mixture was stirred at rt under N₂ for 16 hr. The reaction mixture was added dropwise to cold ether (20 mL) yielding a white precipitate. The precipitate was washed with ether (2×20 mL), dried under N₂ and placed on high vacuum. Yielded light orange hydroscopic solid (370 mg, 75% yield). The product, N,N,N-trimethylaminopropylamine was analyzed by TLC and mass spectrometry (Sciex API 150EX).

### Example 53

Synthesis of N,N,N-trimethylaminopropylamine-CDMNC12 (MC928).

To N,N,N-trimethylaminopropylamine (0.50 mg, 25 μL of 20 μg/μL 75% THF/DMF, 4.3 μmol), was added diisopropylethylamine (0.55 mg, 5.5 μL of 100 μg/μL THF, Aldrich Chemical Company) and vortexed. To the resulting solution was added CDMNC12 (1.5 mg, 15 μL of 100 μg/μL THF) and vortexed. The reaction stirred at rt for 30 min, concentrated on the speed vacuum to afford N,N,N-trimethylaminopropylamine-CDMNC12 (MC928) without further purification. The resulting residue was taken up with DMF (100 μL, 20 μg/μL).

### Example 54

Synthesis of N,N,N-trimethylaminopropyl-dimethyloctadecylsilazane (MC927).

To N,N,N-trimethylaminopropylamine (0.81 mg, 41 μL of 20 μg/μL 75% THF/DMF, 7.0 μmol), was added diisopropyldiethylamine (0.90 mg, 9.0 μL of 100 μg/μL THF, Aldrich Chemical Company). To the resulting solution was added chlorodimethyloctadecylsilane (1.4 mg, 14 μL of 100 μg/μL THF, Aldrich Chemical Company). The reaction was stirred at rt for 30 min, concentrated on the speed vacuum afford N,N,N-trimethylaminopropyl-dimethyloctadecylsilazane (MC927) without further purification. The resulting residue was taken up with DMF (100 μL, 20 μg/μL).

### Example 55

Preparation of the Mixed disulfide of thiopopionic-3-dimethylaminopropanoate and decanethiol (MC746).

To a solution of S-trityl-thiopropionic acid (0.36 g, 1.0 mmol, Aldrich Chemical Company) in dichloromethane (4.0 mL) was added PyBOP (0.54 g, 1.0 mmol, NovaBioChem). The mixture was stirred at ambient temperature for 5 min before the addition of dimethylaminopropanol (0.12 mL, 1.0 mmol, Aldrich Chemical Company). The reaction was continuously stirred at room temperature for 18 hr, and concentrated under reduced pressure. The residue was brought up in EtOAc and partitioned in H₂O. The organic layer was washed 2×H₂O, 1× brine, dried (Na₂SO4), and the solvent removed (aspirator). The resulting residue was suspended in 2% TIPS/50% TFA/CH₂Cl₂ (3 mL) to remove the trityl protecting group. After 2 hr the solution was concentrated to afford thiopopionic-3-dimethylaminopropanoate.

To a solution of thiopopionic-3-dimethylaminopropanoate (0.10 g, 0.52 mmol) in dichloromethane (2 mL) was added decanethiolchloride (0.11 g, 0.52 mmol). The resulting solution was stirred at ambient temperature for 20 min. The solvent was removed and a portion of the resulting residue (25 mg) was purified by plug filtration on silica gel (10% MeOH/CH₂Cl₂ eluent) to afford 20.9 mg (84%) of the mixed disulfide of decanethiol and thiopopionic-3dimethylaminopropanoate (M+1=364.4).

### Example 56

Preparation of the Mixed disulfide of thiopopionic-3-dimethylaminopropanoate and dodecanethiol (MC747).

To a solution of thiopopionic-3-dimethylaminopropanoate (0.10 g, 0.52 mmol) in dichloromethane (2 mL) was added dodecanethiolchloride (0.11 g, 0.52 mmol). The resulting solution was stirred at ambient temperature for 20 min. The solvent was removed and a portion of the resulting residue (150 mg) was purified by flash column chromatography on silica gel (1% TFA/10% MeOH/CH₂Cl₂ eluent) to afford 38 mg (25%) of the mixed disulfide of decanethiol and thiopopionic-3-dimethylaminopropanoate (M+1=392.4).

### Example 57

Amidation of L-Lysine with Laurylamine.

Di-N-Boc-L-Lysine dicyclohexylammonium salt (1.0 g, 1.9 mmol, Sigma Chemical Company) was taken up with EtOAc (50 mL) and partitioned with H₂O (50 mL), washed with HCl (3×50 mL of 1N) and brine (1×50 mL). The organic layer was dried over MgSO₄, filtered and concentrated under reduced pressure. The material was verified by mass spectrometry (Sciex API 150EX) (M+1=347.7). The concentrated material was taken up with dichloromethane (10 mL). To the resulting solution was added dicyclohexylcarbodiimide (0.78 g, 3.8 mmol, Aldrich Chemical Company) and laurylamine (0.4 g, 2.2 mmol, Aldrich Chemical Company). The reaction was covered with a blanket of N₂ and stirred at rt for 1 hr. The reaction was analyzed by TLC to verify that the reaction was complete. The reaction mixture was filtered to remove the DCU and concentrated under reduced pressure. The concentrated material was taken up with 5% MeOH/CH₂Cl₂ (1 mL) and ran on a silica gel column with 5% MeOH/CH₂Cl₂ (100 mL). The fraction was concentrated under reduced pressure and yielded an oil (410 mg). The oil was taken up with TFA/CH₂Cl₂/TIPS (5 mL/2 mL/0.05 mL) and stirred at rt for 1 hr under N₂. The reaction was analyzed by TLC to verify that the reaction was complete. The material was concentrated under reduced pressure and taken up with acetonitrile (1 mL) and precipitated in H₂O. The precipitate was dried under vacuum to afford the laurylamine amide of L-lysine ad off white crystals (77 mg).

### Example 58

Preparation of Pyrenedodecanoic-3-Trimethylaminopropylamine Carboxamide Iodide.

Pyrenedodecanoic acid (10 mg, 0.025 mmol, Molecular Probes) was taken up in THF (0.13 mL, 0.2 M). To the resulting solution was added 3-dimethylaminopropylamine (0.004 mL, 3.2 mg, 0.031 mmol, Aldrich Chemical Company) followed by the addition of dicyclohexylcarbodiimide (6.4 mg, 0.031 mmol, Aldrich Chemical Company). The reactions were stirred at rt under N₂ protected from light for 16 hr. The reaction mixture was analyzed by TLC and mass spectrometry (Sciex API 150EX) to verify that the reaction was complete. The reaction mixture was concentrated under reduced pressure, taken up in EtOAc (0.5 mL), partitioned in H₂O (0.5 mL), washed with HC1 (2×0.5 mL of 1N) and brine (0.5 mL). The organic layer was concentrated under reduced pressure to afford pyrenedodecanoic-3-dimethylaminopropylamine carboxamide.

Pyrenedodecanoic-3-dimethylaminopropylamine carboxamide was taken up in acetonitrile (0.1 mL, 0.25 M). To the resulting solution was added a catalytic amount of K₂CO₃ followed by methyl iodide (0.03 mL, 7.1 mg, 0.050 mmol, Aldrich Chemical Company). The reaction mixture was covered with N₂ and heated (55C.) with stirring for 16 hr. The reaction mixture was analyzed by TLC and mass spectrometry (Sciex API 150EX) to verify that the reaction was complete. The reaction was concentrated under reduced pressure. The concentrated material was taken up in dichloromethane (1 mL), filtered through a plug of silica (6 cm, 10% MeOH/CH₂Cl₂ (50 mL) eluent). The solution was concentrated under reduced pressure, to afford the pyrenedodecanoic-3-trimethylaminopropylamine carboxamide iodide an oil (8.7 mg, 56% yield).

### Example 59

Preparation of FITC-Chit-Ol.

To Chit-Ol (10 mg, 10 mL of 1 mg/mL in 80% DMF solution, 0.026 mmol amine) was added Na₂CO₃ (10 mL of 0.1 M) to raise the pH to 9. Fluorescein isothiocyanate (2 mg, 0.2 mL of 10 mg/mL DMF solution, 0.0053 mmol, Aldrich Chemical Company) was added to the solution. The reaction was stirred at rt and protected from light for 16 hr. The reaction was analyzed by TLC to verify that the reaction was complete. The reaction was concentrated under reduced pressure, precipitated in cold ether (10 mL) and washed with ether (2×10 mL). The material was dried on the high vacuum and reconstituted in H₂O (2 mL, 5 mg/mL). Material (5 mg, 1 mL) was loaded onto a Sephadex G50 column, eluted with H₂O and collected fractions (1 mL). The fractions were analyzed by fluorescence (Varian Cary Eclipse spectrofluorometer, Ex 495 nm and Em 530 nm). Fractions 24-37 were pooled together, froze and lyophilized. Yielded fluffy yellow/orange material (5.0 mg) and was taken up in H₂O (0.5 mL, 10 mg/mL).

### Example 60

Preparation of Rh-DNA-NC₁₂-PyrDet.

Rh-DNA (100 pg, 50 μL of 2 μg/μL H₂O solution, 0.30 μmol phosphate) was taken up in H₂O (50 μL, 1 μg/μL pDNA) and gently mixed. In a separate tube, dodecylamine hydrochloride (13 μg, 13 μL of 10 μg/μL H₂O solution, 0.61 μmol) and PyrDet (9.5 μg, 9.5 μL of 1 μg/μL THF, 0.015 μmol) were mixed together. The detergents were added to the Rh-DNA solution and gently mixed. The material precipitated and was spun down by centrifugation. The supernatant was removed and the pellet was washed with H₂O (2×100 μL). The pellet was lyophilized over P₂O₅ for 72 hr and reconstituted in THF (100 μL, 1 μg/μL).

### Example 61

Ultracentrifugation of Hydrophobic pDNA Complexes.

| Sample | Amt (μg) | H₂O (μL) |
|---|---|---|
| Rh-DNA | 50 | 1000 |
| Rh-DNA-NC₁₂-PyrDet | 50 | 1000 |
| Rh-DNA/FITC-Chit-O1 | 50/105 | 1000 |
| PyrDet | 4.7 | 1000 |
| Rh-DNA-NC₁₂-PyrDet/FITC-Chit-O1 | 50/105 | 1000 |
| FITC-Chit-O1 | 105 | 1000 |

To H₂O (1000 μL) was added either polymer or detergent and vortexed. To the resulting solution was added RhDNA or Rh-DNA-NC₁₂-PyrDet (50 μg) and vortexed. The samples were loaded into tubes prepared with a sucrose gradient (18-54%, 10 mM Hepes, 1 mM EDTA, 9.5 mL) and trizamide as the base (cushion). The samples were centrifugated at 35 krpm at 4C. under vacuum for 20 hr. Each sample was separated into 750 μL fractions, fractionating from the bottom of the tube to the top. To each fraction was added NaCl (150 μL, of 5 M, 1 M final) and Triton (37.5 μL of 20%, 1% final) with vortexing. The fractions were analyzed on a spectrofluorometer (Varian Cary Eclipse) at the rhodamine (Ex 555 nm and Em 585 nm), FITC (Ex 495 nm and Em 530 nm), and pyrene (Ex 340nm and Em 377 nm) channels.

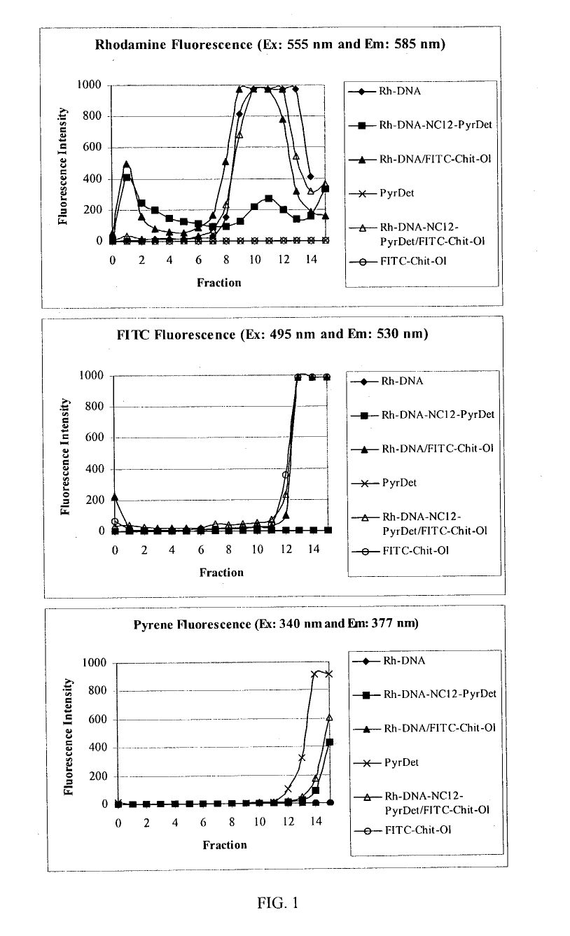

Rhodamine fluorescence results (FIG. 1) indicate that both Rh-DNA-NC₁₂-PyrDet and Rh-DNA/FITC-Chit-Ol form particles/aggregates based on the observed fluorescence of the densest fractions (fractions 12-14). FITC fluorescence results indicates that most of the FITC-Chit-Ol remains in the least dense fractions (top fractions). A small amount of FITC fluorescence is observed in fraction 7 for the complex (Rh-DNA-NC₁₂-PyrDet/FITC-Chit-Ol) and overlaps with the rhodamine fluorescence complex and thus indicates an interaction between the Rh-DNA-NC₁₂-PyrDet with the FITC-Chit-Ol. Pyrene fluorescence results indicate that all pyrene detergent remains in the top fractions. Intensities for the Rh-DNA-NC₁₂-PyrDet and Rh-DNA-NC₁₂-PyrDet/FITC-Chit-Ol are most likely lower as a result of adding PyrDet during formulation of Rh-DNA-NC₁₂-PyrDet. If the PyrDet was not incorporated into the Rh-DNA-NC₁₂ particle, it would have been washed out during the H₂O washes during formulation. This would explain the observed lower fluorescence intensity.

### Example 62

Preparation of Geranylamine Hydrochloride.

Geranlyamine (0.6 mL, 500 mg, 3.3 mmol, Aldrich Chemical Company) was taken up with methanol (0.4 mL) and gently mixed with vortexing. The solution was added dropwise to cold HCl/ether (5.5 mL of 1M, Aldrich Chemical Company) and formed a white precipitate. The precipitate was spun down under centrifugation and washed with ether (2×6 mL). The material was dried under N₂ then on high vacuum. Yielded white crystalline material (550 mg, 89%)

### Example 63

Preparation of pDNA-CPB.

pDNA (pCI Luc, 50 μL,100 μg of 2 μg/μL H₂O, 0.30 μmol phosphate) was added cetylpyridinium bromide (24.4 μL, 244 μg of 10 μg/μL H₂O, 0.61 μmol, Aldrich Chemical Company) and gently mixed. The precipitate was spun down by centrifugation and lyophilized over P₂O₅.

### Example 64

Preparation of pDNA-NC₃ (1:1).

In triplicate, pDNA (pCI Luc, 50 μg, 25 μL of 2 μg/μL H₂O, 0.15 μmol phosphate, pMIR48) was taken up in H₂O (25 μL) and vortexed. To the resulting solution was added NC₃ HCl (1.4 μL, 14 μg of 10 μg/μL H₂O solution, 0.15 μmol, Aldrich Chemical Company) and gently mixed. The solution was froze and dried for 16 hr on the speed vacuum.

### Example 65

Preparation of pDNA-MC927 (1:1).

The dried material, pDNA-NC₃ (50 μg, 0.15 μmol phosphate) was taken up with MC927 (4 μL, 40 μg of 10 μg/μL DMF solution, 0.15 μmol) and vortexed. To the resulting solution was added DMF (21 μL, 2 μg/μL pDNA).

### Example 66

Preparation of pDNA-MC927 (1:2).

The dried material, pDNA-NC₃ (50 μg, 0.15 μmol phosphate) was taken up with MC927 (9 μL, 90 μg of 10 μg/μL DMF solution, 0.30 μmol) and vortexed. To the resulting solution was added DMF (16 μL, 2 μg/μL pDNA).

### Example 67

Preparation of pDNA-MC927 (1:3).

The dried material, pDNA-NC₃ (50 μg, 0.15 μmol phosphate) was taken up with MC927 (13 μL, 130 μg of 10 μg/μL DMF solution, 0.45 μmol) and vortexed. To the resulting solution was added DMF (12 μL, 2 μg/μL pDNA).

### Example 68

Preparation of pDNA-MC933 (1:1).

pDNA (pCI Luc, 50 μg, 25 μL of 2 μg/μL solution, 0.15 μmol phosphate) was taken up in H₂O (25 μL) and vortexed. To the resulting solution was added MC933 (7 μL, 70 μg of 10 μg/μL H₂O solution pH 8, 0.15 μmol) and gently mixed. The solution was froze and dried for 16 hr on the speed vacuum.

### Example 69

Preparation of pDNA-MC933 (1:2).

pDNA (pCI Luc, 50 μg, 25 μL of 2 μg/μL H₂O, 0.15 μmol phosphate) was taken up in H₂O (25 μL) and vortexed. To the resulting solution was added MC933 (15 μL, 150 μg of 10 μg/μL H₂O solution pH 8, 0.30 μmol) and gently mixed. The solution was froze and dried for 16 hr on the speed vacuum.

By a similar procedure as described for MC933 (1:2) the following preparations were formed: pDNA-NC₆, pDNA-NC₁₀, pDNA-CHAPS, pDNA-Lys-NC12, pDNA-MC753, pDNA-MC754, pDNA-MC744, pDNA-MC745, pDNA-MC746, pDNA-MC747, pDNA-MC909.

### Example 70

Preparation of pDNA-MC933 (1:3).

pDNA (pCI Luc, 50 μg, 25 μL of 2 μg/μL solution, 0.15 μmol phosphate) was taken up in H₂O (25 μL) and vortexed. To the resulting solution was added MC933 (220 μL, 22 μg of 10 μg/μL H₂O solution pH 8, 0.45 μmol) and gently mixed. The solution was frozen and dried 16 hr on the speed vacuum.

### Example 71

Preparation of Ol-Mel-CDM.

Ol-Mel (40 μL, 400 μg of 10 μg/μL DMF solution, 0.52 μmol amine) was added diisopropylethylamine (1.8 μL, 18 μg of 10 μg/μL DMF solution, 0.14 μμol, Aldrich Chemical Company) and vortexed. CDM (2.6 μL, 26 μg of 10 μg/μL DMF solution, 0.14 μmol) was added and vortexed. The reaction mixture was vortexed at rt for 30 min. Final concentration was at 10 μg/μL Ol-Mel.

### Example 72

Preparation of Ol-Mel-CDM(2.5).

Ol-Mel (25 μL, 250 μg of 10 μg/μL DMF solution, 0.32 μmol amine) was added diisopropylethylamine (1.1 μL, 11 μg of 10 μg/μL DMF solution, 0.085 μμol, Aldrich Chemical Company) and vortexed. CDM (3.7 μL, 37 μg of 10 μg/μL DMF solution, 0.20 μmol) was added and vortexed. The reaction mixture was vortexed at rt for 30 min. Final concentration was at 8.4 μg/μL Ol-Mel.

### Example 73

Hepa Cell Transfection.

10% FBS and 24 hr Harvest (starting confluency 55%). Seventeen Samples were formulated as follows:

1) To Opti (300 μL) was added pDNA (pCI Luc, 1.5 μL, 3 μg of 2 μg/μL H₂O) and vortexed. Lt-1 (9 μL, Mirus Corporation) was added and vortexed.

2-7) To H₂O (300 μL) was added pDNA-MC927 formulations (pCI Luc, 1.5 μL, 3 μg of 2 μg/μL DMF solution) and vortexed.

8) To H₂O (300 μL) was added pDNA (pCI Luc, 1.5 μL, 3 μg of 2 μg/μL H₂O) and vortexed. Ol-Mel-CDM (1.5 μL, 15 μg of 10 μg/μL DMF solution) was added and vortexed.

9) To H₂O (300 μL) was added pDNA (pCI Luc, 1.5 μL, 3 μg of 2 μg/μL H₂O) and vortexed. Ol-Mel-CDM (3 μL, 30 μg of 10 μg/μL DMF solution) was added and vortexed.

10-11) To H₂O (300 μL) was added pDNA-MC927 (pCI Luc, 1.5 μL, 3 μg of 2 μg/μL DMF solution) and vortexed. Ol-Mel-CDM (1.5 μL, 15 μg of 10 μg/μL DMF solution) was added and vortexed.

12-13) To H₂O (300 μL) was added pDNA-MC927 (pCI Luc, 1.5 μL, 3 μg of 2 μg/μL DMF solution) and vortexed. Ol-Mel-CDM (3 μL, 30 μg of 10 μg/μL DMF solution) was added and vortexed.

14-15) To H₂O (300 μL) was added pDNA-MC933 (pCI Luc, 1.5 μL, 3 μg of 2 μg/μL DMF solution) and vortexed. Ol-Mel-CDM (1.5 μL, 15 μg of 10 μg/μL DMF solution) was added and vortexed.

16-17) To H₂O (300 μL) was added pDNA-MC933 (pCI Luc, 1.5 μL, 3 μg of 2 μg/μL DMF solution) and vortexed. Ol-Mel-CDM (3 μL, 30 μg of 10 μg/μL DMF solution) was added and vortexed.

Hepa cells were maintained in DMEM. Approximately 24 h prior to transfection, cells were plated at an appropriate density in 48-well plates and incubated overnight. Cultures were maintained in a humidified atmosphere containing 5% CO2 at 37° C. The cells were transfected at a starting confluency of 73% by combining 100 μL sample (1 μg pDNA per well) with the cells in 1 mL of media. Cells were harvested after 24 h and assayed for luciferase activity using a Lumat LB 9507 (EG&G Berthold, Bad-Wildbad, Germany) luminometer. The amount of luciferase expression was recorded in relative light units. Numbers are the average for two separate wells.

Hepa Cell Transfection Results:

| # | Complex | wt Ratio (μg) | RLU Mean |
|---|---|---|---|
| 1 | pDNA + Lt-1 | 1/3 μL | 16,024,685 |
| 2 | pDNA-MC927 (1:1) | 1 | 103 |
| 3 | pDNA-MC927 (1:2) | 1 | 96 |
| 4 | pDNA-MC927 (1:3) | 1 | 81 |
| 5 | pDNA-MC933 (1:1) | 1 | 91 |
| 6 | pDNA-MC933 (1:2) | 1 | 77 |
| 7 | pDNA-MC933 (1:3) | 1 | 121 |
| 8 | pDNA + O1-Mel-CDM | 1/5 | 76,320 |
| 9 | pDNA + O1-Mel-CDM | 1/10 | 637,307 |
| 10 | pDNA-MC927(1:1) + O1-Mel-CDM | 1/5 | 56,349 |
| 11 | pDNA-MC927(1:2) + O1-Mel-CDM | 1/5 | 81,021 |
| 12 | pDNA-MC927(1:1) + O1-Mel-CDM | 1/10 | 9,007 |
| 13 | pDNA-MC927(1:2) + O1-Mel-CDM | 1/10 | 29,503 |
| 14 | pDNA-MC933(1:1) + O1-Mel-CDM | 1/5 | 149,091 |
| 15 | pDNA-MC933(1:2) + O1-Mel-CDM | 1/5 | 594,391 |
| 16 | pDNA-MC933(1:1) + O1-Mel-CDM | 1/10 | 213,776 |
| 17 | pDNA-MC933(1:2) + O1-Mel-CDM | 1/10 | 98,898 |

Results indicate that the pDNA/cationic surfactant complex is expressible in vitro when interacted with the ol-mel derivatives.

### Example 74

Hepa Cell Transfection.

10% FBS, 24 hr harvest.

Twenty-three samples were formulated for cell transfections.

1) To Opti (200 μL) was added pDNA (pCI Luc, 1 μL, 2 μg of 2 μg/μL H₂O) and the solution was vortexed. Lt-1 (6 μL, Mirus Corporation) was added and the solution was again vortexed.

2-25) To H₂O (200 μL) was added pDNA or pDNA/cationic surfactant complex (pCI Luc, 1 μL, 2 μg of 2 μg/μL DMF) and the solution was vortexed. Ol-Mel or Ol-Mel-CDM was added and the solution was again vortexed.

Hepa cells were maintained in DMEM. Approximately 24 h prior to transfection, cells were plated at an appropriate density in 48-well plates and incubated overnight. Cultures were maintained in a humidified atmosphere containing 5% CO2 at 37° C. The cells were transfected at a starting confluency of 73% by combining 100 μL sample (1 μg pDNA per well) with the cells in 1 mL of media. Cells were harvested after 24 h and assayed for luciferase activity using a Lumat LB 9507 (EG&G Berthold, Bad-Wildbad, Germany) luminometer. The amount of luciferase expression was recorded in relative light units. Numbers are the average for two separate wells.

| | | wt Ratio | |
|---|---|---|---|
| # | Complex | (μg) | RLU Mean |
| 1 | pDNA + Lt-1 | 1/3 μL | 18,598,949 |
| 2 | pDNA + O1-Mel | 1/5 | 22,822 |
| 3 | pDNA + O1-Mel | 1/10 | 68,954 |
| 4 | pDNA + O1-Mel | 1/15 | 2,686 |
| 5 | pDNA-MC933(1:2) + O1-Mel | 1/5 | 481,520 |
| 6 | pDNA-MC933(1:2) + O1-Mel | 1/10 | 193,520 |
| 7 | pDNA-MC933(1:2) + O1-Mel | 1/15 | 4,876 |
| 8 | pDNA + O1-Mel-CDM | 1/5 | 12,917 |
| 9 | pDNA + O1-Mel-CDM | 1/10 | 119,281 |
| 10 | pDNA + O1-Mel-CDM | 1/15 | 12,310 |
| 11 | pDNA-MC927(1:2) + O1-Mel-CDM | 1/5 | 64,128 |
| 12 | pDNA-MC927(1:2) + O1-Mel-CDM | 1/10 | 86,395 |
| 13 | pDNA-MC927(1:2) + O1-Mel-CDM | 1/15 | 1,409 |
| 14 | pDNA-MC933(1:2) + O1-Mel-CDM | 1/5 | 424,049 |
| 15 | pDNA-MC933(1:2) + O1-Mel-CDM | 1/10 | 298,644 |
| 16 | pDNA-MC933(1:2) + O1-Mel-CDM | 1/15 | 9,358 |
| 17 | pDNA + O1-Mel-CDM(2.5) | 1/5 | 20,570 |
| 18 | pDNA + O1-Mel-CDM(2.5) | 1/10 | 78,689 |
| 19 | pDNA + O1-Mel-CDM(2.5) | 1/15 | 9,137 |
| 20 | pDNA-MC927(1:2) + O1-Mel-CDM(2.5) | 1/5 | 69,485 |
| 21 | pDNA-MC927(1:2) + O1-Mel-CDM(2.5) | 1/10 | 111,287 |
| 22 | pDNA-MC927(1:2) + O1-Mel-CDM(2.5) | 1/15 | 14,218 |
| 23 | pDNA-MC933(1:2) + O1-Mel-CDM(2.5) | 1/5 | 722,841 |
| 24 | pDNA-MC933(1:2) + O1-Mel-CDM(2.5) | 1/10 | 129,665 |
| 25 | pDNA-MC933(1:2) + O1-Mel-CDM(2.5) | 1/15 | 5,611 |

Results indicate that the pDNA/cationic surfactant complex is expressible in vitro when interacted with the modified peptide derivatives.

### Example 75

Preparation of GL3-153-NC,₂.

To H₂O (46.2 μL) was added GL3-153 (3.8 μL, 50 μg of 13.3 μg/μL 10 mM NaCl, 0.16 μmol phosphate, 2′OH—CWU ACG CUG AGU ACU UCG AdTdT (SEQ ID 12) and its compliment 2′OH-UCG AAG UAC UCA GCG UAA GdTdT (SEQ ID 13), Dharmacon) and vortexed. To the resulting solution was added dodecylamine HCl (7 μL, 70μg, of 10 μg/μL H₂O, 0.31 μmol) and gently mixed. The reaction mixture was incubated at rt for 30 min, froze and lyophilized over P₂O₅ 16 hr. The dried material was reconstituted in DMF (25 μL, 2 μg/μL GL3-153)

### Example 76

Preparation of EGFP-64-NC₁₂.

To H₂O (46.3 μL) was added EGFP-64 (3.7 μL, 50 μg of 13.4 μg/μL 10 mM NaCl, 0.16 μmol phosphate, 2′OH-GAC GUA AAC GGC CAC AAG UGC AdTdT (SEQ ID 14) and its compliment 2′OH—CG CUG CAU UUG CCG GUG UUC A GdTdT (SEQ ID 15, Dharmacon) and vortexed. To the resulting solution was added dodecylamine HCl (7 μL, 70 μg, of 10 μg/μL H₂O, 0.31 μmol) and gently mixed. The reaction mixture was incubated at rt for 30 min, froze and lyophilized over P₂O₅ 16 hr. The dried material was reconstituted in DMF (25 μL, 2 μg/μL EGFP-64)

### Example 77

Mouse Tail Vein Injections.

HP Tail Vein Dual Luciferase: siRNA delivery Five complexes were prepared as follows:

Complex I-V: To Ringers (10 mL, 1×) was added pMIR116 (pCI Luc, 40 μg, 20 μL of 2 μg/μL H₂O) and vortexed. pMIR122 (pCI Ren, 4 μg, 2 μL of 2 μg/μL H₂O) was added to the solution and vortexed.

Complex II: To the plasmid solution was added GL3-153 (20 μg, 1.5 u1L of 13.3 μg/μL 10 mM NaCl, Dharmacon) and vortexed.

Complex III: To the plasmid solution was added EGFP-64 (20 μg, 1.5 μL of 13.4 μg/μL 10 mM NaCl, Dharmacon) and vortexed.

Complex IV: To the plasmid solution was added GL3-153-NC₁₂ (20 μg, 10 μL of 2 μg/μL DMF) and vortexed.

Complex V: To the plasmid solution was added EGFP-64-NC₁₂ (20 μg, 10 μL of 2 μg/μL DMF) and vortexed.

Tail vein injections of 1.0 mL per 10 g body weight (˜2.5 ml) were preformed on ICR mice (n=2) using a 30 gauge, 0.5 inch needle. Injections were done manually with injection times of 4-5 sec [Zhang et al. 1999; Liu et al. 1999]. One day after injection, the livers were harvested and homogenized in lysis buffer (0.1% Triton X-100, 0.1 M K-phosphate, 1 mM DTT, pH 7.8). Insoluble material was cleared by centrifugation and 10 μl of the cellular extract or extract diluted 10× was analyzed for luciferase activity as previously reported [Wolffetal 1990].

Results:

| Complex # | Formulation | Luc RLU | Ren RLU | Luc/Ren |
|---|---|---|---|---|
| Complex I | pMIR116/pMIR122 | 12,986,650 | 42,206,060 | 31 |
| Complex I | pMIR116/pMIR122 | 56,986,540 | 153,988,270 | 37 |
| Complex II | pMIR116/pMIR122/GL3-153 | 16,886,310 | 62,075,320 | 27 |
| Complex II | pMIR116/pMIR122/GL3-153 | 12,171,210 | 165,623,300 | 7 |
| Complex III | pMIR116/pMIR122/EGFP-64 | 1,730,890 | 41,515,930 | 4 |
| Complex III | pMIR116/pMIR122/EGFP-64 | 6,513,560 | 128,718,130 | 5 |
| Complex IV | pMIR116/pMIR122/GL3-153- | 49,854,070 | 143,555,100 | 35 |
| | NC12 | | | |
| Complex IV | pMIR116/pMIR122/GL3-153- | 13,225,000 | 26,978,760 | 49 |
| | NC12 | | | |
| Complex V | pMIR116/pMIR122/EGFP-64- | 22,132,210 | 57,071,140 | 39 |
| | NC12 | | | |
| Complex V | pMIR116/pMIR122/EGFP-64- | 6,517,230 | 113,748,060 | 6 |
| | NC12 | | | |

Results indicate that the hydrophobic siRNA (GL3-153) inhibits luciferase expression.

### Example 78

Preparation of CDM-Mel-Si(Me)₂C₁₈.

Melittin (15 μL, 300 μg, of 20 μg/μL DMF, 0.11 μmol) was taken up in DMF (2.5 μL) and vortexed. To the resulting solution was added diisopropylethylamine (1.5 μL, 15 μg of 10 μg/μL DMF, 0.11 μmol, Aldrich Chemical Company) and vortexed. Chlorodimethyloctadecylsilane (4 μL, 40 μg of 10 μg/μL THF, 0.11 μmol, Aldrich Chemical Company) was added to the solution and vortexed. The reaction was heated (70° C.) for 5 min and diisopropylethylamine (2 μL, 20 μg of 10 μg/μL DMF, 0.16 μmol, Aldrich Chemical Company) was added and vortexed. CDM (5 μL, 50 μg of 10 μg/μL DMF, 0.27 μmol) was added and vortexed. The reaction mixture was mixed with shaking for 30 min at rt. The resulting mixture was used without purification.

### Example 79

Preparation of CDM-Mel.

Melittin (15 μL, 300 μg, of 20 μg/μL DMF, 0.11 μmol) was taken up in DMF (6.5 μL) and vortexed. To the resulting solution was added diisopropylethylamine (3.5 μL, 35 μg of 10 μg/μL DMF, 0.27 μmol, Aldrich Chemical Company) and vortexed. CDM (5 μL, 50 μg of 10 μg/μL DMF, 0.27 μmol) was added and vortexed. The reaction mixture was mixed with shaking for 30 min at rt. The resulting mixture was used without purification.

### Example 80

Preparation of CDM-Mel-CDMNC12.

Melittin (15 82 L, 300 μg, of 20 μg/μL DMF, 0.11 μmol) was taken up in DMF (3.5 μL) and vortexed. To the resulting solution was added diisopropylethylamine (1.5 μL, 15 μg of 10 μg/μL DMF, 0.11 μmol, Aldrich Chemical Company) and vortexed. CDMNC12 (4 μL, 40 μg of 10 μg/μL DMF, 0.11 μmol) was added and vortexed. The reaction mixture was mixed with shaking for 30 min at rt. To the reaction mixture was added diisopropylethylamine (2 μL, 20 μg of 10 μg/μL DMF, 0.16 μmol, Aldrich Chemical Company) and vortexed. CDM (5 μL, 50 μg of 10 μg/μL DMF, 0.27 μmol) was added to the reaction and vortexed. The reaction was mixed with shaking for 30 min at rt. The resulting mixture was used without purification.

### Example 81

Mouse Tail Vein Injections HP.

Six complexes were made as follows:

Complex I: To H₂O (9.7 mL) was added pDNA-NC₁₂ (pCI Luc, 20 μL, 40 μg of 2 μg/μL DMF) and vortexed. CDM-Mel-SiMe₂C₁₈ (15 μL, 150 μg of 10 μg/μL DMF) was added immediately before injection.

Complex II: To H₂O (9.7 mL) was added pDNA-NC₁₂ (pCI Luc, 20 μL, 40 μg of 2 μg/μL DMF) and vortexed. Ol-Mel-CDM (15 μL, 150 μg of 10 μg/μL DMF) was added and vortexed.

Complex III: To H₂O (9.7 mL) was added pDNA-NC₁₂ (pCI Luc, 20 μL, 40 μg of 2 μg/μL DMF) and vortexed. CDM-Mel-CDMNC12 (15 μL, 150 μg of 10 μg/μL DMF) was added and vortexed.

Complex IV: To H₂O (9.7 mL) was added pDNA-NC₁₂ (pCI Luc, 20 μL, 40 μg of 2 μg/μL DMF) and vortexed. CDM-Mel (15 μL, 150 μg of 10 μg/μL DMF) was added and vortexed.

Complex V: To H₂O (9.7 mL) was added pDNA-NC₁₂ (pCI Luc, 20 μL, 40 μg of 2 μg/μL DMF) and vortexed.

Complex VI: To H₂O (9.7 mL) was added pDNA (pCI Luc, 20 μL, 40 μg of 2 μg/μL H₂O) and vortexed. NaCl (300 μL of 5 M, 150 mM final) was added to each complex prior to injection. Tail vein injections of 1.0 mL per 10 g body weight (˜2.5 ml) were preformed on ICR mice (n=2) using a 30 gauge, 0.5 inch needle. Injections were done manually with injection times of 4-5 sec [Zhang et al. 1999; Liu et al. 1999]. One day after injection, the livers were harvested and homogenized in lysis buffer (0.1% Triton X-100, 0.1 M K-phosphate, 1 mM DTT, pH 7.8). Insoluble material was cleared by centrifugation and 10 μl of the cellular extract or extract diluted 10× was analyzed for luciferase activity as previously reported [Wolff et al 1990].

Results: Mean RLU (n=3)

| | Liver 100× | | | | |
|---|---|---|---|---|---|
| Complex # | dilution | Spleen | Lung | Heart | Kidney |
| Complex I | 3,268,842 | 704,378 | 1,553,333 | 216,703 | 471,391 |
| Complex II | 1,395,436 | 144,766 | 176,723 | 37,294 | 757,639 |
| Complex III | 6,000,523 | 646,471 | 843,741 | 115,154 | 579,530 |
| Complex IV | 791,962 | 197,011 | 78,403 | 26,309 | 74,368 |
| Complex V | 2,425,812 | 125,634 | 201,128 | 65,457 | 250,216 |
| Complex VI | 1,753,888 | 140,723 | 211,857 | 209,652 | 599,696 |

### Example 82

Low Pressure Tail Vein Mouse.

Four complexes were made as follows:

Complex I: To H₂O (600 μL) was added pDNA-NC₁₂ (pCI Luc, 15 μL, 30 μg of 2 μg/μL DMF) was added and vortexed. Immediately before injection, CDM-Mel-SiMe₂C₁₈ (11.2 μL, 112 μg of 10 μg/μL DMF) was added and vortexed.

Complex II: To H₂O (600 μL) was added pDNA-NC₁₂ (pCI Luc, 15 μL, 30 μg of 2 μg/μL DMF) was added and vortexed. Ol-Mel-CDM (11.2 μL, 112 μg of 10 μg/μL DMF) was added and vortexed.

Complex III: To H₂O (600 μL) was added pDNA-NC₁₂ (pCI Luc, 15 μL, 30 μg of 2 μg/μL DMF) was added and vortexed. CDM-Mel-CDMNC12 (11.2 μL, 112 μg of 10 μg/μL DMF) was added and vortexed.

Complex IV: To H₂O (600 μL) was added pDNA-NC₁₂ (pCI Luc, 15 μL, 30 μg of 2 μg/μL DMF) was added and vortexed. CDM-Mel (11.2 μL, 112 μg of 10 μg/μL DMF) was added and vortexed. 200 μL tail vein injections of 200 μL of the complex were preformed on ICR mice (n=2) using a 30 gauge, 0.5 inch needle, with the total solution injected by hand within 10 seconds. One day after injection, the animal was sacrificed, and a luciferase assay was conducted on the liver, spleen, lung, heart, and kidney. Luciferase expression was determined as previously reported (Wolff, J. A., Malone, R. W., Williams, P., Chong, W., Acsadi, G., Jani, A. and Felgner, P. L. Direct gene transfer into mouse muscle in vivo. Science, 1465-1468, 1990.). A Lumat LB 9507 (EG&G Berthold, Bad-Wildbad, Germany) luminometer was used.

Results: Mean RLU (n=2)

| Complex # | Liver | Spleen | Lung | Heart | Kidney |
|---|---|---|---|---|---|
| Complex I | 1,155,276 | 1,879 | 1,071 | 1,339 | 3,733 |
| Complex II | 51,508 | 3,287 | 474 | 954 | 2,400 |
| Complex III | 907 | 2,504 | 860 | 274 | 632 |
| Complex IV | 1,685 | 416 | 320 | 247 | 292 |

The results indicate that hydrophobic modified peptide ternary complexes deliver pDNA predominatly to the liver, where the pDNA is able to be transcribed.

### Example 83

Bile Duct Injections.

Two complexes were made as follows:

Complex I: To isotonic mannitol (800 μL) was added pDNA-MC933 (1:2) (pCI Luc, 20 μL, 40 μg of 2 μg/μL DMF) and vortexed.

Complex II: To isotonic mannitol (800 μL) was added pDNA-MC933 (1:2) (pCI Luc, 20 μL, 40 μg of 2 μg/μL DMF) and vortexed. Ol-Mel-CDM (15 μL, 150 μg of 10 μg/μL DMF) was added and vortexed.

Bile duct injections on ICR mice were performed using a Harvard Apparatus PH 2000 programmable pump with a 30-gauge, ½ inch needle and 1 ml syringe. The pump was programmed to deliver 200 μL over 4 seconds. A 5×1 mm, Kleinert Kutz microvessel clip was used to occlude the bile duct downstream from the point of injection in order to prevent flow to the duodenum and away from the liver. The gallbladder inlet was not occluded. In these injections, the junction of the hepatic vein and caudal vena cava were not clamped. Additonally, the portal vein and hepatic artery were not clamed for the injection.

Results: Mean RLU

| Complex # | n = | Liver |
|---|---|---|
| Complex I | 3 | 403,002 |
| Complex II | 2 | 508,318 |

The results indicate that the described binary and ternary complexes are able to deliver pDNA to the liver via the bile duct.

### Example 84

Bile Duct Injections.

Four Complexes were made as follows:

Complex I: To isotonic mannitol (800 μL) was added pDNA-NC₁₂ (pCI Luc, 20 μL, 40 μg of 2 μg/μL DMF) and vortexed. Ol-Mel-CDM (15 μL, 150 μg of 10 μg/μL DMF) was added and vortexed.

Complex II: To isotonic mannitol (800 μL) was added pDNA-NC₁₂ (pCI Luc, 20 μL, 40 μg of 2 μg/μL DMF) and vortexed. Ol-Mel-CDMC12 (15 μL, 150 μg of 10 μg/4L DMF) was added and vortexed.

Complex III: To isotonic mannitol (800 μL) was added pDNA-NC₁₂ (pCI Luc, 20 μL, 40 μg of 2 μg/μL DMF) and vortexed. CDM-Mel (15 μL, 150 μg of 10 μg/μL DMF) was added and vortexed.

Complex IV: To isotonic mannitol (800 μL) was added pDNA-NC₁₂ (pCI Luc, 20 μL, 40 μg of 2 μg/μL DMF) and vortexed. Ol-Mel-CDM (15 μL, 150 μg of 10 μg/μL DMF) was added and vortexed. MC894 (3.5 μL, 70 μg of 20 μg/μL DMF) was added and vortexed.

Bile duct injections on ICR mice were performed using a Harvard Apparatus PH 2000 programmable pump with a 30-gauge, ½ inch needle and 1 ml syringe. The pump was programmed to deliver 200 μL over 4 seconds. A 5×1 mm, Kleinert Kutz microvessel clip was used to occlude the bile duct downstream from the point of injection in order to prevent flow to the duodenum and away from the liver. The gallbladder inlet was not occluded. In these injections, the junction of the hepatic vein and caudal vena cava were not clamped. Additonally, the portal vein and hepatic artery were not clamed for the injection.

Results: Mean RLU (n=3)

| Complex # | Liver 10× Diluted |
|---|---|
| Complex I | 76,485 |
| Complex II | 31,771 |
| Complex III | 91,396 |
| Complex IV | 92,118 |

The results indicate that the described binary and ternary complexes are able to deliver pDNA to the liver via the bile duct.

### Example 85

pDNA-NC₁₂ Degradation with DNAse I via Gel Electrophoresis.

Samples were formulated (2-13) as described below (total volume 10 μL) and incubated at 37° C. for 30 min. Loading buffer was added to all samples and mixed. The samples were loaded onto 0.8% agarose gel containing ethidium bromide. The gel was run at 95V for 45 min and analyzed by UV light.

| Lane | Description |
|---|---|
| 1 | 1 kb Ladder (500 ng) |
| 2 | pDNA (500 ng) |
| 3 | pDNA (500 ng) + DNase I (1.25 u) |
| 4 | pDNA (500 ng) + DNase I (2.5 u) |
| 5 | pDNA (500 ng) + DNase I (5 u) |
| 6 | pDNA (500 ng) + pLL (315 ng) |
| 7 | pDNA (500 ng) + pLL (315 ng) + |
| | DNase I (1.25 u) |
| 8 | pDNA (500 ng) + pLL (315 ng) + |
| | DNase I (2.5 u) |
| 9 | pDNA (500 ng) + pLL (315 ng) + |
| | DNase I (5 u) |
| 10 | pDNA-NC₁₂ (500 ng) |
| 11 | pDNA-NC₁₂ (500 ng) + DNase I |
| | (1.25 u) |
| 12 | pDNA-NC₁₂ (500 ng) + DNase I |
| | (2.5 u) |
| 13 | pDNA-NC₁₂ (500 ng) + DNase I |
| | (5 u) |
| 14 | empty |

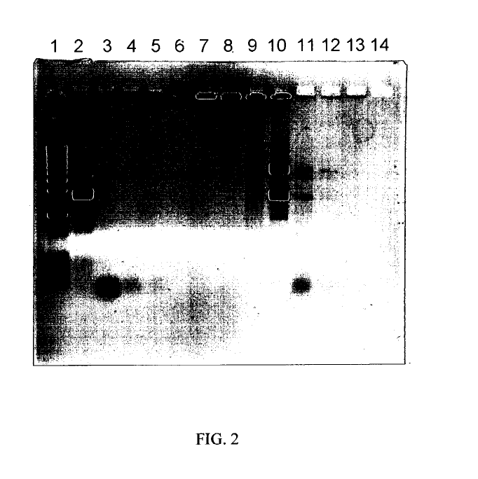

Results (FIG. 2) indicate that the pDNA in the pDNA-NC₁₂ complex is still accessible to DNAseI.

### Example 86

Condensation of RhDNA with NC₁₂ Concentration and Temperature Dependence.

Five complexes were made in quadruplicate as follows:

Complex I: To H₂O (500 μL) was added RhDNA (2.5 μL, 5 μg of 2 μg/μL H₂O, 0.015 μmol) and vortexed. Dodecylamine hydrochloride (6.7 μL, 6.7 μg, of 1 μg/μL H₂O, 0.030 μmol) was added to the solution and vortexed. The final concentration of RhDNA was 0.1 μg/μL.

Complex II: To H₂O (468 μL) was added RhDNA (25 μL, 50 μg of 2 μg/μL H₂O, 0.15 μmol) and vortexed. Dodecylamine hydrochloride (6.7 μL, 67 μg, of 10 μg/μL, 0.030 μmol) was added to the solution and vortexed. The final concentration of RhDNA was 0.1 μg/μL.

Complex III: To H₂O (218 μL) was added RhDNA (25 μL, 50 μg of 2 μg/μL H₂O, 0.15 μmol) and vortexed. Dodecylamine hydrochloride (6.7 μL, 67 μg, of 10 μg/μL, 0.030 μmol) was added to the solution and vortexed. The final concentration of RhDNA was 0.2 μg/μL.

Complex IV: RhDNA-NC₁₂ (2 μg/μL DMF) Complex IV: RhDNA-NC₁₂ (2 μg/μL benzyl alcohol)

Complexes I-III were incubated at different temperatures (RT, 37° C., 50° C. and 70° C.). Their fluorescence (Varian Cary Eclipse Spectrofluorometer) was monitored at several time points (t=0, 1, 2, 4 and 20 hr) and compared to Complexes IV and V.

Results: The results indicate that the pDNA in the binary complex is more condensed when the sample is heated relative to a room temperature sample. The more dilute samples indicate the greatest amount of condensation relative to time, and the closer in overall condensation to controls that were dried under vacumn.

### Example 87

NaCl Stability of RhDNA+NC₁₂ Complexes.

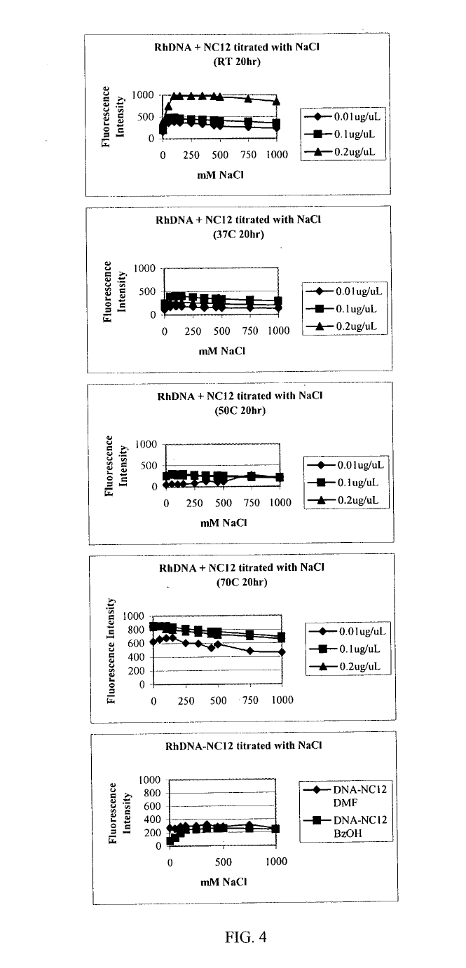

After the 20 hr incubation, samples were titrated with NaCl and analyzed by fluorescence (Varian Cary Eclipse Spectrofluorometer). FIG. 4. Five complexes were made in quadruplicate as follows:

Complex I: To H₂O (500 μL) was added RhDNA (2.5 μL, 5 μg of 2 μg/μL H₂O, 0.015 μmol) and vortexed. Dodecylamine hydrochloride (6.7 μL, 6.7 μg, of 1 μg/μL H₂O, 0.030 μmol) was added to the solution and vortexed. The final concentration of RhDNA was 0.1 μg/μL.

Complex II: To H₂O (468 μL) was added RhDNA (25 μL, 50 μg of 2 μg/μL H₂O, 0.15 μmol) and vortexed. Dodecylamine hydrochloride (6.7 μL, 67 μg, of 10 μg/μL, 0.030 mol) was added to the solution and vortexed. The final concentration of RhDNA was 0.1 μg/μL.

Complex III: To H₂O (218 μL) was added RhDNA (25 μL, 50 μg of 2 μg/μL H₂O, 0.15 μmol) and vortexed. Dodecylamine hydrochloride (6.7 μL, 67 μg, of 10 μg/μL, 0.030 μmol) was added to the solution and vortexed. The final concentration of RhDNA was 0.2 μg/μL.

Complex IV: RhDNA-NC₁₂ (2 μg/μL DMF)

Complex IV: RhDNA-NC₁₂ (2 μg/μL benzyl alcohol)

Complexes I-III were incubated at several different temperatures (RT, 37° C., 50° C. and 70° C.). After the 20 hr incubation, samples were titrated with NaCl and analyzed by fluorescence (Varian Cary Eclipse Spectrofluorometer). FIG. 4

Results. The results indicate that the sample heated to 70° C. decondenses in the presence of NaCl. The 50° C. sample is stable in salt, and the 37° C. indicates a small amount of decondensation, whereas the rt samples indicate greater decondensation. For the 37° C. and RT samples, the more dilute the sample, the greater the stability, indicating that the binary complex can be stabilized based on time, temperature, and concentration.

### Example 88

RhDNA-NC₁₂ Stability at 37° C. with 150 mM NaCl.

Four complexes were made in duplicate as follows:

Complex I: To H₂O (500 μL) was added RhDNA-NC₁₂ (2.5 μL, 5 μg of 2 μg/μL DMF, 0.015 μmol) and vortexed.

Complex II: To H₂O (500 μL) was added RhDNA (2.5 μL, 5 μg of 2 μg/μL H₂O, 0.015 μmol) and vortexed. Dodecylamine hydrochloride (6.7 μL, 6.7 μg, of 1 μg/μL H₂O, 0.030 μmol) was added to the solution and vortexed.

Complex III: To H₂O (500 μL) was added RhDNA (2.5 μL, 5 μg of 2 μg/μL H₂O, 0.015 μmol) and vortexed. p-L-Lysine HBr (9.5 μL, 9.5 μg, of 1 μg/μL H₂), 0.030 μmol, Sigma Chemical Company) was added to the solution and vortexed.

Complex IV: To H₂O (500 μL) was added RhDNA (2.5 μL, 5 μg of 2 μg/μL H₂O, 0.015 μmol) and vortexed

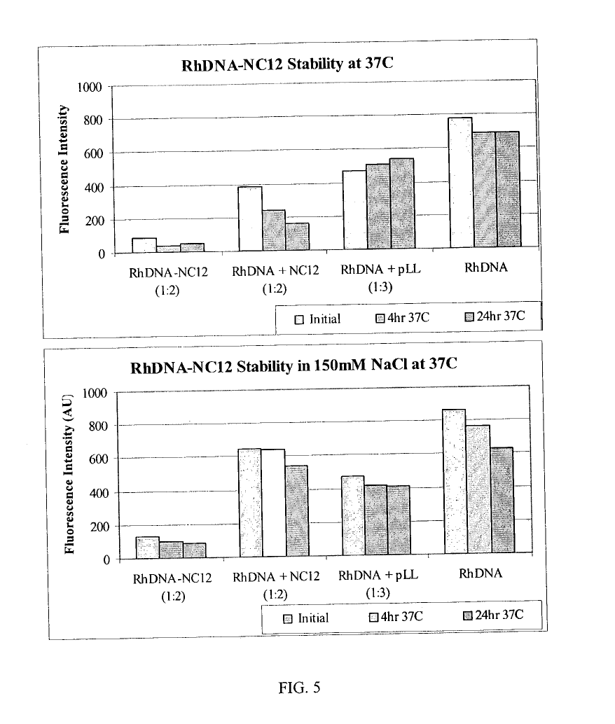

To one set of the complexes, NaCl (15 μL of 5M, 150 mM) was added to each complex and the solutions were vortexed. To the second set of complexes, no NaCl was added. All complexes were incubated at 37C. The fluorescence of each sample was analyzed over several hours (t=0, 4, 24 hr) (Varian Cary Eclipse Spectrofluorometer). FIG. 5. Results: The results indicate that samples heated in the presence of salt show less condensation of the pDNA, than samples heated with no salt present.

### Example 89

RhDNA-NC₁₂ Stability in Serum.

Three complexes were made as follows:

Complex I: To H₂O (500 μL) was added RhDNA-NC₁₂ (2.5 μL, 5 μg of 2 μg/μL DMF, 0.015 μmol) and vortexed.

Complex II: To H₂O (500 μL) was added RhDNA (2.5 μL, 5 μg of 2 μg/μL H₂O, 0.015 μmol) and vortexed. Dodecylamine hydrochloride (6.7 μL, 6.7 μg, of 1 μg/μL H₂O, 0.030 μmol) was added to the solution and vortexed.

Complex III: To H₂O (500 μL) was added RhDNA (2.5 μL, 5 μg of 2 μg/μL H₂O, 0.015 μmol) and vortexed. p-L-Lysine HBr (9.5 μL, 9.5 μg, of 1 μg/μL H₂O, 0.030 μmol, Sigma Chemical Company) was added to the solution and vortexed.

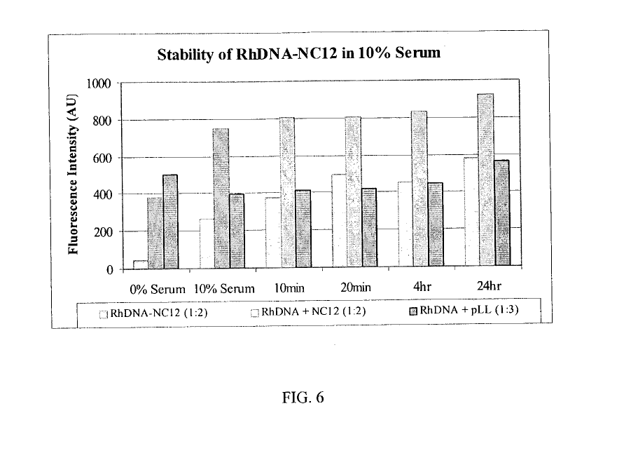

Samples were analyzed by fluorescence (Varian Cary Eclipse Spectrofluorometer). Serum (5 μL, 10%) was added to each complex with vortexing. The fluorescence of each sample was analyzed over several hours (t=0, 0.2, 0.3, 4, 24 hr) (Varian Cary Eclipse Spectrofluorometer). FIG. 6

Results: The results indicate that serum causes a decrease in the level of condensation over time, indicated that the pDNA is being released in the presence of serum over time.

### Example 90

Circular Dichroism of Hydrophobic DNA.

Preparation of pDNA+NC₁₂+37C. (0.2 μg/μL): pDNA (pCI Luc, 100 μL, 200 μg of 2 μg/μL, 0.61 μmol) was taken up in H₂O (873 μL) and vortexed. To the resulting solution was added dodecylamine hydrochloride (27 μL, 270 μg of 10 μg/μL H₂O, 1.2 μmol). The solution was incubated at 37C. for 16 h. Four complexes were made as follows:

Complex I: To H₂O (1955 μL) was added pDNA (pCI Luc, 30 μL, 60 μg of 2 μg/μL H₂O) and vortexed. Methanol (15 μL) was added to the solution and vortexed.

Complex II: To H₂O (1947 μL) was added pDNA (pCI Luc, 30 μL, 60 μg of 2 μg/μL H₂O) and vortexed. Dodecylamine hydrochloride (8 μL, 80 μg of 10 μg/μL H₂O) was added to the solution and vortexed. Methanol (15 μL) was added to the solution and vortexed.

Complex III: To H₂O (1685 μL) was added pDNA+NC₁₂+37C. (300 μL, 60 μg of 0.2 μg/μL H₂O) and vortexed. Dodecylamine hydrochloride (8 μL, 80 μg of 10 μg/μL H₂O) was added to the solution and vortexed. Methanol (15 μL) was added to the solution and vortexed

Complex IV: To H₂O (1985 μL) was added pDNA-NC₁₂ (15 82 L, 60 μg of 4 μg/μL methanol) and vortexed.

Results: The results indicate that there is a shift in the CD spectra in the presence of a cationic detergent. The results also indicate a lowering of the signal intensity for a complex that has been dried.

### Example 91

Liposomal Incorporation of a Binary Complex.

Rhodamine labeled pDNA/lauryl amine hydrochloride binary complex (RhDNA-NC12) was prepared as previously described, and the dried solid was dissolved in DMF as a 2 mg/mL solution. To phosphatidylcholine (Avanti Polar Lipids, Ins.)/cholesterol hemisuccinate (Sigma Chemical Company) (2 mg, 1:1 wt, in chloroform) was added 20 μg of RhDNA-NC12 (based on DNA wt). The solution was dried under reduced pressure and then under high vacuum. The resulting residue was hydrated with 2 mL water, and sonicated to form liposomes. The solution was passed through a size exclusion column (sepharose CL-4B-200, 150 mM NaCl eluent, 1 mL fractions).

Results: The results indicate that the RhDNA eluded with the liposome fractions, and separate from RhDNA blank.

### Example 92

In Vivo Tail Vein Injections into Mice.

Complexes were made as follows:

Complex I: To H₂O (1600 μL) was added pDNA-NC₁₂ (pCI Luc, 32 μL, 80 μg of 2.5 μg/μL benzyl alcohol) was added and vortexed.

Complex II: To H₂O (1600 μL) was added Chit-Ol-LBA (168 μg, 252 μL of 0.67 μg/μL DMF/H₂) and vortexed. pDNA-NC₁₂ (pCI Luc, 32 μL, 80 μg of 2.5 μg/μL benzyl alcohol) was added and vortexed.

Complex III: To H₂O (1600 μL) was added Chit-Ol-LBA (168 μg, 252 μL of 0.67 μg/μL DMF/H₂) and vortexed. pDNA-NC₆ (pCI Luc, 32 μL, 80 μg of 2.5 μg/μL benzyl alcohol) was added and vortexed.

200 μL tail vein injections of 200 μL of the complex were preformed on ICR mice (n=4) using a 30 gauge, 0.5 inch needle, with the total solution injected by hand within 10 seconds. Three days after injection, the animal from group A was sacrificed, and a luciferase assay was conducted on the liver, spleen, lung, and kidney. Seven days after injection, the animal from group B was sacrificed, and a luciferase assay was conducted on the liver, spleen, lung, heart, and kidney. Luciferase expression was determined as previously reported (Wolff et al. 1990.). A Lumat LB 9507 (EG&G Berthold, Bad-Wildbad, Germany) luminometer was used.

Results: Mean RLU (n=2) Harvest Day 3, Group A

| Complex # | Liver | Spleen | Lung | Kidney |
|---|---|---|---|---|
| Complex I | 20,599 | 433 | 521 | 512 |
| Complex II* | 5,306 | 471 | 449 | 679 |
| Complex III* | 4,517 | 336 | 686 | 347 |

*n = 1

Results: Mean RLU (n=2) Harvest Day 7, Group B

| Complex # | Liver | Spleen | Lung | Kidney |
|---|---|---|---|---|
| Complex I | 2,783 | 3,803 | 4,068 | 3,105 |
| Complex II* | 943 | 1,670 | 1,299 | 2,373 |
| Complex III* | 1,597 | 1,667 | 1,407 | 1,507 |

*n = 1

The results indicate that the pDNA in the pDNA-NC12 complexes is accessible for transcription.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

References:

1. Legendre, J. & Szoka, F. Delivery of plasmid DNA into mammalian cell lines using pH-sensitive liposomes: Comparison with cationic liposomes. Pharmaceut. Res. 9, 1235-1242(1992)

2. Kamata, H., Yagisawa, H., Takahashi, S. & Hirata, H. Amphiphilic peptides enhance the efficiency of liposome-mediated DNA transfection. Nucleic Acids Res. 22, 536-537 (1994). Wagner, E., Curiel, D. & Cotten, M. Delivery of drugs, proteins and genes into cells using transferrin as a ligand for receptor-mediated endocytosis. Advanced Drug Delivery Reviews 14, 113-135 (1994)

3. Duzgunes, N., Straubinger, R. M., Baldwin, P. A. & Papahadjopoulos, D. PH-sensitive liposomes. (eds Wilsehub, J. & Hoekstra, D.) p. 713-730 (Marcel Deker INC, 1991)

4. Melnikov, 1995

5. Sergeyev, 1999

6. Sergeyev, 1999

7. Sukhorukov, 2000

8. Tanaka, 1996

9. Ijiro, 1992

10. Melnikov, 1995

11. Clamme J P, Bemacchi S, Vuilleumier C, Duportail G, Mely Y. Gene transfer by cationic surfactants is essentially limited by the trapping of the surfactant/DNA complexes onto the cell membrane: a fluorescence investigation. Biochim Biophys Acta. Aug. 25, 2000;1467(2):347-61.

12. Trubetskoy V S, et al. (1999). “Quantitative assessment of DNA condensation.” Anal Biochem 267(2): 309-313.

13. Wolff, J. A., Malone, R. W., Williams, P., Chong, W., Aesadi, G., Jani, A. and Felgner, P. L. Direct gene transfer into mouse muscle in vivo. Science, 1465-1468, 1990.

14. Zhang, G., Vargo, D., Budker, V., Armstrong, N., Knechtle, S., Wolff, J. A. Human Gene Therapy, 8, 1763-1772, 1997.

15. Zhang et al. 1999

16. Liu et al. 1999

17. Calbiochem, 2000-2001

We claim:

1. A process for delivering a polynucleotide to a cell in vivo comprising: associating the polynucleotide with a cationic surfactant to form a binary complex, stabilizing the complex, and delivering the complex to a cell in a mammal.

2. The process of claim 1 wherein the cationic surfactant consists of a detergent.

3. The process of claim 1 wherein stabilizing the complex comprises: incubating the complex at elevated temperature.

4. The process of claim 1 wherein stabilizing the complex consists of drying the complex.

5. The process of claim 1 wherein delivering the complex to a cell in a mammal comprises injecting the complex in a solution into a tissue in a mammal.

6. The process of claim 1 wherein delivering the complex to a cell in a mammal comprises inserting the complex in a solution into a vessel in a mammal.

7. The process of claim 6 wherein the vessel is selected from the listing consisting of: portal vein, hepatic vein, inferior vena cava, tail vein, hepatic artery, and bile duct.

8. The process of claim 1 wherein the cell consists of a liver cell.

9. The process of claim 8 wherein the liver cell consists of a hepatocyte.

10. The process of claim 1 wherein the polynucleotide consists of DNA.

11. The process of claim 10 wherein the DNA comprises an expression cassette.

12. The process of claim 11 wherein the expression cassette encodes a protein.

13. The process of claim 1 wherein the polynucleotide consists of a polynucleotide containing 18-30 nucleotide monomeric subunits.

14. The process of claim 13 wherein the polynucleotide consists of a polynucleotide that induces RNA interference.

15. A process for delivering a polynucleotide to a cell comprising: associating a polynucleotide with a cationic surfactant to from a binary complex, stabilizing the binary complex, associating the binary complex with an amphipathic compound to form a ternary complex, and delivering the complex to the cell.

16. The process of claim 15 wherein the cationic surfactant is selected from the list consisting of: detergent and lipid.

17. The process of claim 15 wherein stabilizing the binary complex comprises: incubating the complex at elevated temperature.

18. The process of claim 15 wherein stabilizing the binary complex consists of drying the complex.

19. The process of claim 15 wherein the amphipathic compound is selected from the list consisting of: polymer containing one or more hydrophobic moieties, peptide containing one or more hydrophobic moieties, targeting group containing one or more hydrophobic moieties, steric stabilizer containing one or more hydrophobic moieties, surfactants and lipids.

20. The process of claim 19 wherein the polymer containing one or more hydrophobic moieties consists of modified chitosan.

21. The process of claim 15 wherein delivering the complex to a cell in a mammal comprises injecting the complex in a solution into a tissue in a mammal.

22. The process of claim 15 wherein delivering the complex to a cell in a mammal comprises inserting the complex in a solution into a vessel in a mammal.

23. The process of claim 22 wherein the vessel is selected from the listing consisting of: portal vein, hepatic vein, inferior vena cava, tail vein, hepatic artery, and bile duct.

24. The process of claim 15 wherein the cell consists of a liver cell.

25. The process of claim 24 wherein the liver cell consists of a hepatocyte.

26. The process of claim 15 wherein the polynucleotide consists of DNA.

27. The process of claim 26 wherein the DNA comprises an expression cassette.

28. The process of claim 27 wherein the expression cassette encodes a protein.

29. The process of claim 15 wherein the polynucleotide consists of a polynucleotide containing 18-30 nucleotide monomeric subunits.

30. The process of claim 29 wherein the polynucleotide consists of a polynucleotide that induces RNA interference.

31. A process for forming a small salt stable polynucleotide-containing complex comprising: associating the polynucleotide with a cationic surfactant in a solution and incubating the complex in the solution at elevated temperature.

32. The process of claim 31 wherein the cationic surfactant is selected from the list consisting of: detergent and lipid.

33. A process for forming a small salt stable polynucleotide-containing complex comprising: associating the polynucleotide with a cationic surfactant in a solution to form a binary complex, stabilizing the binary complex, and associating the binary complex with an amphipathic compound to form a ternary complex.

34. The process of claim 34 wherein the cationic surfactant is selected from the list consisting of: detergent and lipid.

35. The process of claim 34 wherein stabilizing the binary complex comprises: incubating the complex at elevated temperature.

36. The process of claim 34 wherein stabilizing the binary complex consists of drying the complex.

37. The process of claim 34 wherein the amphipathic compound is selected from the list consisting of: polymer containing one or more hydrophobic moieties, peptide containing one or more hydrophobic moieties, targeting group containing one or more hydrophobic moieties, steric stabilizer containing one or more hydrophobic moieties, detergent and lipid.

38. The process of claim 38 wherein the polymer containing one or more hydrophobic moieties consists of modified chitosan.


SEQUENCE LISTING (15 sequences)

SEQ ID NO: 1 - protein, 13 aa, Simian virus 40
CGYGPKKKRKVGG

SEQ ID NO: 2 - protein, 39 aa, Simian virus 40
CKKKSSSDDEATADSQHSTPPKKKRKVEDPKDFPSELLS

SEQ ID NO: 3 - protein, 37 aa, Simian virus 40
CKKKWDDEATADSQHSTPPKKKRKVEDPKDFPSELLS

SEQ ID NO: 4 - protein, 31 aa, Homo sapiens
CYNDFGNYNNQSSNFGPMKQGNFGGRSSGPY

SEQ ID NO: 5 - protein, 10 aa, Human adenovirus type 1
CKRGPKRPRP

SEQ ID NO: 6 - protein, 22 aa, Xenopus laevis
CKKAVKRPAATKKAGQAKKKKL

SEQ ID NO: 7 - protein, 14 aa, Homo sapiens
CKKKGPAAKRVKLD

SEQ ID NO: 8 - protein, 4 aa, Homo sapiens
KDEL

SEQ ID NO: 9 - protein, 10 aa, Human immunodeficiency virus
GRKKRRQRRR

SEQ ID NO: 10 - protein, 16 aa, Drosophila melanogaster
RQIKIWFQNRRMKWKK

SEQ ID NO: 11 - protein, 10 aa, Artificial
  polyarginine synthetic peptide
CRRRRRRRRR

SEQ ID NO: 12 - DNA, 21 bp, Photinus pyralis
cuuacgcugaguacuucgatt

SEQ ID NO: 13 - DNA, 21 bp, Photinus pyralis
ucgaaguacucagcguaagtt

SEQ ID NO: 14 - DNA, 24 bp, Aequorea victoria
gacguaaacggccacaagugcatt

SEQ ID NO: 15 - DNA, 24 bp, Aequorea victoria
cgcugcauuugccgguguucagtt